SEQUENCE LISTING (16 sequences)

SEQ ID NO: 1 - DNA, 22 bp, Homo sapiens
tgttctacacggaaccctcctc

SEQ ID NO: 2 - DNA, 21 bp, Homo sapiens
ctgggcaaatgccaatgagat

SEQ ID NO: 3 - DNA, 22 bp, Homo sapiens
ttctcctgccctactctcccac

SEQ ID NO: 4 - DNA, 22 bp, Homo sapiens
caggctggttattgaaaccttg

SEQ ID NO: 5 - DNA, 25 bp, Homo sapiens
ccctaaggaaaaaacaactaaacgc

SEQ ID NO: 6 - DNA, 20 bp, Homo sapiens
ctgccatgctcatccccaga

SEQ ID NO: 7 - DNA, 23 bp, Homo sapiens
cttgttccttcttttctcctgcc

SEQ ID NO: 8 - DNA, 22 bp, Homo sapiens
gttttacattcaaggccgtcag

SEQ ID NO: 9 - DNA, 21 bp, Homo sapiens
caactaaacgccgctttcctg

SEQ ID NO: 10 - DNA, 22 bp, Homo sapiens
aagctgacgttcttggcactgc

SEQ ID NO: 11 - DNA, 23 bp, Homo sapiens
gctttcctgcctcaggatgaact

SEQ ID NO: 12 - DNA, 22 bp, Homo sapiens
ccttggcaggattgaagctgac

SEQ ID NO: 13 - DNA, 24 bp, Homo sapiens
gatgaactgacggccttgaatgta

SEQ ID NO: 14 - DNA, 23 bp, Homo sapiens
cctggcagagttgtctcaccttg

SEQ ID NO: 15 - DNA, 6985 bp, Homo sapiens
attgtccgatggttcccggcgtacctcggcttccctcggtagtttccggcaatggtcgagagtttctaacgtgcccccttgttgtctctcggccgccgtcctctcaaccaccgccccccttttcggctccctctcccccttcccgttcccccagtcagcctggccctgctggtgcctccggcgctacgggctgggcaagatggcggccttcgggatcttgagctacgaacaccggcccctgaagcggccgcggctggggcctcccgatgtttaccctcaggaccccaaacagaaggaggatgaactgacggccttgaatgtaaaacaaggtttcaataaccagcctgctgtctctggggatgagcatggcagtgccaagaacgtcagcttcaatcctgccaagatcagttccaacttcagcagcattattgcagagaaattacgttgtaatacccttcctgacactggtcgcaggaagccccaagtgaaccagaaggataacttctggctggtgactgcacgatcccagagtgccattaacacttggttcactgacttggctggcaccaagccactcacgcaactagccaaaaaggtccccattttcagtaagaaggaagaggtgtttgggtacttagccaaatacacagtgcctgtgatgcgggctgcctggctcattaagatgacctgtgcctactatgcagcaatctctgagaccaaggttaagaagagacatgttgaccctttcatggaatggactcagatcatcaccaagtacttatgggagcagttacagaagatggctgaatactaccggccagggcctgcaggaagtgggggctgtggttccacgatagggcccttgccccatgatgtagaggtggcaatccggcagtgggattacaccgagaagctggccatgttcatgtttcaggatggaatgctggacagacatgagttcctgacctgggtgcttgagtgttttgagaagatccgccctggagaggatgaattgcttaaactgctgctgcctctgcttctccgatactctggggaatttgttcagtctgcatacctgtcccgccggcttgcctacttctgtacacggagactggccctgcagctggatggtgtgagcagtcactcatctcatgttatatctgctcagtcaacaagcacgctacccaccacccctgctcctcagcccccaactagcagcacaccctcgactccctttagtgacctgcttatgtgccctcagcaccggcccctggtttttggcctcagctgtatcctacagaccatcctcctgtgctgtcctagtgccttggtttggcactactcactgactgatagcagaattaagaccggctcaccacttgaccacttgcctattgccccgtccaacctgcccatgccagagggtaacagtgccttcactcagcaggtccgtgcaaagttgcgggagatcgagcagcagatcaaggagcggggacaggcagttgaagttcgctggtctttcgataaatgccaggaagctactgcaggcttcaccattggacgggtacttcatactttggaagtgctggacagccatagttttgaacgctctgacttcagcaactctcttgactccctttgtaaccgaatctttggattgggacctagcaaggatgggcatgagatctcctcagatgatgatgctgtggtgtcattgctatgtgaatgggctgtcagctgcaagcgttctggtcggcatcgtgctatggtggtagccaagctcctggagaagagacaggcggagattgaggctgagcgttgtggagaatcagaagccgcagatgagaagggttccatcgcctctggctccctttctgctcccagtgctcccattttccaggatgtcctcctgcagtttctggatacacaggctcccatgctgacggaccctcgaagtgagagtgagcgggtggaattctttaacttagtactgctgttctgtgaactgattcgacatgatgttttctcccacaacatgtatacttgcactctcatctcccgaggggaccttgcctttggagcccctggtccccggcctccctctccctttgatgatcctgccgatgacccagagcacaaggaggctgaaggcagcagcagcagcaagctggaagatccagggctctcagaatctatggacattgaccctagttccagtgttctctttgaggacatggagaagcctgatttctcattgttctcccctactatgccctgtgaggggaagggcagtccatcccctgagaagccagatgtcgagaaggaggtgaagcccccacccaaggagaagattgaagggacccttggggttctttacgaccagccacgacacgtgcagtacgccacccattttcccatcccccaggaggagtcatgcagccatgagtgcaaccagcggttggtcgtactgtttggggtgggaaagcagcgagatgatgcccgccatgccatcaagaaaatcaccaaggatatcttgaaggttctgaaccgcaaagggacagcagaaactgaccagcttgctcctattgtgcctctgaatcctggagacctgacattcttaggtggggaggatgggcagaagcggcgacgcaaccggcctgaagccttccccactgctgaagatatctttgctaagttccagcacctttcacattatgaccaacaccaggtcacggctcaggtctcccggaatgttctggagcagatcacgagctttgcccttggcatgtcataccacttgcctctggtgcagcatgtgcagttcatcttcgacctcatggaatattcactcagcatcagtggcctcatcgactttgccattcagctgctgaatgaactgagtgtagttgaggctgagctgcttctcaaatcctcggatctggtgggcagctacactactagcctgtgcctgtgcatcgtggctgtcctgcggcactatcatgcctgcctcatcctcaaccaggaccagatggcacaggtctttgaggggctgtgtggcgtcgtgaagcatgggatgaaccggtccgatggctcctctgcagagcgctgtatccttgcttatctctatgatctgtacacctcctgtagccatttaaagaacaaatttggggagctcttcagcgacttttgctcaaaggtgaagaacaccatctactgcaacgtggagccatcggaatcaaatatgcgctgggcacctgagttcatgatcgacactctagagaaccctgcagctcacaccttcacctacacggggctaggcaagagtcttagtgagaaccctgctaaccgctacagctttgtctgcaatgcccttatgcacgtctgtgtggggcaccatgatcccgatagggtgaatgacatcgcaatcctgtgtgcagagctgaccggctattgcaagtcactgagtgcagaatggctaggagtgcttaaggccttgtgctgctcctctaacaatggcacttgtggtttcaacgatctcctctgcaatgttgatgtcagtgacctatcttttcatgactcgctggctacttttgttgccatcctcatcgctcggcagtgtttgctcctggaagatctgattcgctgtgctgccatcccttcactccttaatgctgcttgtagtgaacaggactctgagccaggggcccggcttacctgccgcatcctccttcaccttttcaagacaccgcagctcaatccttgccagtctgatggaaacaagcctacagtaggaatccgctcctcctgcgaccgccacctgctggctgcctcccagaaccgcatcgtggatggagccgtgtttgctgttctcaaggctgtgtttgtacttggggatgcggaactgaaaggttcaggcttcactgtgacaggaggaacagaagaacttccagaggaggagggaggaggtggcagtggtggtcggaggcagggtggccgcaacatctctgtggagacagccagtctggatgtctatgccaagtacgtgctgcgcagcatctgccaacaggaatgggtaggagaacgttgccttaagtctctgtgtgaggacagcaatgacctgcaagacccagtgttgagtagtgcccaggcgcagcgcctcatgcagctcatttgctatccacatcgactgctggacaatgaggatggggaaaacccccagcggcagcgcataaagcgcattctccagaacttggaccagtggaccatgcgccagtcttccttggagctgcagctcatgatcaagcagacccctaacaatgagatgaactccctcttggagaacatcgccaaggccacaatcgaggttttccaacagtcagcagagacagggtcatcttctggaagtactgcaagcaacatgcccagcagcagcaagaccaagcctgtgctcagctctctagagcgctctggtgtatggctggtggcccccctcattgctaaactgcccacctcagtccagggacatgtgttaaaggctgctggggaagaattggagaagggtcagcacctgggttcctcttcacgcaaagaacgtgatcgacaaaagcagaagagcatgtccctattgagccagcagcccttcttatcgctggtgctaacatgtctgaaagggcaggatgaacaacgcgagggactccttacctccctctacagccaggtgcaccagattgtgaataattggcgagatgaccagtacttagatgattgcaaaccaaagcagcttatgcatgaggcactcaaactgcggctcaacctggtggggggcatgtttgacacggtgcagcgcagcacccagcagaccacggagtgggccatgctcctcctggagatcatcatcagcggcactgtcgacatgcagtccaacaatgagctcttcactactgtgttggacatgctgagcgtgctcatcaatgggacattggctgcagacatgtctagcatctcgcaaggtagcatggaggaaaacaagcgtgcatacatgaacctggcgaagaagttgcagaaggagttgggggagcgccagtcagacagtctggaaaaggttcgccagctgctgccactgcccaagcagacccgagatgtcatcacgtgtgagccacagggctcccttatcgataccaagggcaacaagattgctggcttcgattccatcttcaagaaggagggtctacaggtttccaccaaacagaagatctcgccctgggatctttttgaggggttgaagccgtcagcaccactctcttggggctggtttggaacagtccgagtggaccggcgagtggctcgaggagaggagcagcagcggttgctgctctaccacacacacctgaggccccggccccgcgcctattacctggagccactgccactgcccccagaagatgaggagccgcctgctcctaccctgctagagcctgagaaaaaggctccagagccccccaaaactgacaaaccgggggctgctccacccagtactgaggaacgcaagaagaagtccaccaagggcaagaaacgcagccagccagctaccaagacagaggactatggaatgggcccgggtcggagcggcccttatggtgtgacagtgcctccggacctcctgcaccacccaaaccctggttctataacacaccttaactacaggcaaggctccataggcctgtacacccagaaccagccactacctgcaggtggccctcgtgtggacccataccgtcctgtgcgcttaccaatgcagaagctgcccacccgaccaacttaccctggagtgctgcccacaaccatgactggcgtcatgggtttagaaccctcctcttataagacctctgtgtaccggcagcagcaacctgcggtgccccaaggacagcgccttcgccaacagctccagcagagtcagggcatgttgggacagtcatctgtccatcagatgactcccagctcttcctacggtttgcagacttcccagggctatactccttatgtttctcatgtgggattgcagcaacacacaggccctgcaggtaccatggtgccccccagctactccagccagccttaccagagcacccacccttctaccaatcctactcttgtagatcctacccgccacctgcaacagcggcccagtggctatgtgcaccagcaggcccccacctatggacatggactgacctccactcaaaggttttcacaccagacactgcagcagacacccatgataagtaccatgactccaatgagtgcccagggcgtccaggcaggcgtccgttcaacagccatcctacctgagcagcagcagcagcagcaacagcagcaacagcaacagcagcagcagcagcaacagcaacagcagcagcagcagcagcagtaccacatccggcagcagcagcagcagcagatcctgcggcagcagcagcaacagcaacagcagcagcagcagcagcagcaacagcaacagcagcagcagcaacagcaacaacagcaacaccagcagcaacagcagcaacaggcggctcctccccaaccccagccccagtcccagccccagttccagcgccaggggcttcagcagacccagcagcagcaacagacagcagctttggtccggcaacttcaacaacagctctctaatacccagccacagcccagtaccaacatatttggacgctactgagccacctggaggaactgcttgtgcactggatgtggccccaccctttcctcttaattcccaatcccattcctgggctagcaccagtagtggttggggccctcccctcaggctccatttttaataagtttttagtatttttgttaatgtgaggcattgagctgttgggttttgtatattatttatatagagaccccagagctgttgcacccaatacacagagcttctttgcaaagggaaaaaaaaaaaaaaaaa

SEQ ID NO: 16 - protein, 2177 aa, Homo sapiens
MAAFGILSYEHRPLKRPRLGPPDVYPQDPKQKEDELTALNVKQGFNNQPAVSGDEHGSAKNVSFNPAKISSNFSSIIAEKLRCNTLPDTGRRKPQVNQKDNFWLVTARSQSAINTWFTDLAGTKPLTQLAKKVPIFSKKEEVFGYLAKYTVPVMRAAWLIKMTCAYYAAISETKVKKRHVDPFMEWTQIITKYLWEQLQKMAEYYRPGPAGSGGCGSTIGPLPHDVEVAIRQWDYTEKLAMFMFQDGMLDRHEFLTWVLECFEKIRPGEDELLKLLLPLLLRYSGEFVQSAYLSRRLAYFCTRRLALQLDGVSSHSSHVISAQSTSTLPTTPAPQPPTSSTPSTPFSDLLMCPQHRPLVFGLSCILQTILLCCPSALVWHYSLTDSRIKTGSPLDHLPIAPSNLPMPEGNSAFTQQVRAKLREIEQQIKERGQAVEVRWSFDKCQEATAGFTIGRVLHTLEVLDSHSFERSDFSNSLDSLCNRIFGLGPSKDGHEISSDDDAVVSLLCEWAVSCKRSGRHRAMVVAKLLEKRQAEIEAERCGESEAADEKGSIASGSLSAPSAPIFQDVLLQFLDTQAPMLTDPRSESERVEFFNLVLLFCELIRHDVFSHNMYTCTLISRGDLAFGAPGPRPPSPFDDPADDPEHKEAEGSSSSKLEDPGLSESMDIDPSSSVLFEDMEKPDFSLFSPTMPCEGKGSPSPEKPDVEKEVKPPPKEKIEGTLGVLYDQPRHVQYATHFPIPQEESCSHECNQRLVVLFGVGKQRDDARHAIKKITKDILKVLNRKGTAETDQLAPIVPLNPGDLTFLGGEDGQKRRRNRPEAFPTAEDIFAKFQHLSHYDQHQVTAQVSRNVLEQITSFALGMSYHLPLVQHVQFIFDLMEYSLSISGLIDFAIQLLNELSVVEAELLLKSSDLVGSYTTSLCLCIVAVLRHYHACLILNQDQMAQVFEGLCGVVKHGMNRSDGSSAERCILAYLYDLYTSCSHLKNKFGELFSDFCSKVKNTIYCNVEPSESNMRWAPEFMIDTLENPAAHTFTYTGLGKSLSENPANRYSFVCNALMHVCVGHHDPDRVNDIAILCAELTGYCKSLSAEWLGVLKALCCSSNNGTCGFNDLLCNVDVSDLSFHDSLATFVAILIARQCLLLEDLIRCAAIPSLLNAACSEQDSEPGARLTCRILLHLFKTPQLNPCQSDGNKPTVGIRSSCDRHLLAASQNRIVDGAVFAVLKAVFVLGDAELKGSGFTVTGGTEELPEEEGGGGSGGRRQGGRNISVETASLDVYAKYVLRSICQQEWVGERCLKSLCEDSNDLQDPVLSSAQAQRLMQLICYPHRLLDNEDGENPQRQRIKRILQNLDQWTMRQSSLELQLMIKQTPNNEMNSLLENIAKATIEVFQQSAETGSSSGSTASNMPSSSKTKPVLSSLERSGVWLVAPLIAKLPTSVQGHVLKAAGEELEKGQHLGSSSRKERDRQKQKSMSLLSQQPFLSLVLTCLKGQDEQREGLLTSLYSQVHQIVNNWRDDQYLDDCKPKQLMHEALKLRLNLVGGMFDTVQRSTQQTTEWAMLLLEIIISGTVDMQSNNELFTTVLDMLSVLINGTLAADMSSISQGSMEENKRAYMNLAKKLQKELGERQSDSLEKVRQLLPLPKQTRDVITCEPQGSLIDTKGNKIAGFDSIFKKEGLQVSTKQKISPWDLFEGLKPSAPLSWGWFGTVRVDRRVARGEEQQRLLLYHTHLRPRPRAYYLEPLPLPPEDEEPPAPTLLEPEKKAPEPPKTDKPGAAPPSTEERKKKSTKGKKRSQPATKTEDYGMGPGRSGPYGVTVPPDLLHHPNPGSITHLNYRQGSIGLYTQNQPLPAGGPRVDPYRPVRLPMQKLPTRPTYPGVLPTTMTGVMGLEPSSYKTSVYRQQQPAVPQGQRLRQQLQQSQGMLGQSSVHQMTPSSSYGLQTSQGYTPYVSHVGLQQHTGPAGTMVPPSYSSQPYQSTHPSTNPTLVDPTRHLQQRPSGYVHQQAPTYGHGLTSTQRFSHQTLQQTPMISTMTPMSAQGVQAGVRSTAILPEQQQQQQQQQQQQQQQQQQQQQQQQQQYHIRQQQQQQILRQQQQQQQQQQQQQQQQQQQQQQQQQQHQQQQQQQAAPPQPQPQSQPQFQRQGLQQTQQQQQTAALVRQLQQQLSNTQPQPSTNIFGRY

(19) United States
(12) Patent Application Publication
(10) Pub. No.: US 2017/0145504 A1
(43) Pub. Date: May 25, 2017

(54) BREAST FIBROADENOMA SUSCEPTIBILITY MUTATIONS AND USE THEREOF

(71) Applicants: Patrick Boon Ooi TAN, Singapore (SG); Puay Hoon TAN, Singapore (SG); Bin Tean TEH, Singapore (SG)

(72) Inventors: Patrick Boon Ooi TAN, Singapore (SG); Puay Hoon TAN, Singapore (SG); Bin Tean TEH, Singapore (SG)

(21) Appl. No.: 15/310,302

(22) PCT Filed: May 12, 2015
(86) PCT No.: PCT/SG2015/050107

§ 371 Date: Nov. 10, 2016

(30) Foreign Application Priority Data
May 12, 2014 (SG) 10201402277X

(51) Int. Cl. C12Q 1/68

(52) U.S. Cl. CPC C12Q 1/6883; C12Q 2600/156

(57) ABSTRACT

The present disclosure provides a method of assaying susceptibility and/or confirming diagnosis of breast fibroadenomas development in a human subject. Preferably, the method comprises the steps of performing a nucleic acid-based assay to analyze an isolated polynucleotide encoding at least exon 2 of MED12 gene from a sample acquired from the human subject; and regarding the human subject with greater susceptibility and/or confirming diagnosis of breast fibroadenomas development by detecting a mutation in the isolated polynucleotide. The mutation can be a splice site mutation located at position −8 of exon 2 of the MED12 gene, a missense mutation located at codon 44 of cDNA of the MED12 gene or a missense mutation located at codon 36 of cDNA of the MED12 gene.

### TECHNICAL FIELD

The present disclosure relates to a method of assaying the risk or susceptibility of fibroadenoma development in a human subject. The disclosed method can be or at least form part of the diagnosis to confirm the occurrence of fibroadenoma in the subject. More particularly, the disclosed method sets out to detect one or more mutations residing within the MED12 gene of the subject that presence of these mutations has shown significant association with the occurrence of fibroadenomas in a human subject.

### BACKGROUND

Fibroadenomas (FAs) are benign breast tumors that represent the most frequently occurring breast tumors in women under the age of 30 years^{1,2}. Often observed in adolescent girls and young adult women, fibroadenomas are clinically known to be hormone-dependent and to fluctuate in size according to periods of pregnancy and menopause³. A study of 265,402 women in China reported a fibroadenoma incidence of 241 per 100,000 among women under 35 years and 165 per 100,000 among women of age 35-39 years⁴. Histologically, fibroadenomas comprise an admixture of stromal and epithelial cells5. Although benign, fibroadenomas are reported to be associated with an approximately two-fold increase in risk of developing invasive breast carcinoma in 20 years⁶. The diagnosis of fibroadenoma is typically achieved by biopsy, and patients with larger lesions are often subjected to surgery which can incur cost, anxiety and in rare cases, procedure-related complications. At present, little is known about the genetic abnormalities that underlie fibroadenoma particularly when compared to breast carcinoma, where much recent progress has been made in the characterization of its mutational landscape^{8,9}. For example, previous targeted mutational screens of TP53 in fibroadenomas have been equivocal. One study reported that one out of eight (12.5%) fibroadenomas exhibited a non-silent TP53 mutation¹⁰, whereas another study reported no somatic TP53 mutations in fibroadenomas from women who remained unaffected by breast cancer after an average follow-up of ten years¹¹. A single PIK3CA mutation has also been reported from a screen of ten fibroadenoma tumors¹². Based upon the findings of these studies, it appears that certain genetic makeups may inevitably predispose an individual to greater risk of FAs development. Therefore, early identification of such genetic attributes or methods allowing one to discover the likelihood of genetic deficiencies is greatly desired.

### SUMMARY

The present disclosure aims to provide a method of assaying the risk of breast fibroadenomas occurrence in a human subject, preferably a female subject, through genotyping a specific allele or gene of the subject.

Still, an object of the present disclosure is to bring forth a method capable of serving as confirming diagnosis or forming at least part of the confirming diagnosis towards the occurrence of fibroadenoma in a human subject.

A further object of the present disclosure is to offer a method of assaying susceptibility and/or confirming diagnosis of fibroadenoma development in a human subject by detecting one or more mutations located in the MED12 gene using any genotyping approaches known in the art.

Another object of the present disclosure is to provide a method of detecting mutations resulting particularly in non-synonymous substitution in the encoded mediator complex subunit 12 (MED12). The mutations to be detected are associated with higher risk of fibroadenomas occurrence in a female subject.

At least one of the preceding objects is met, in whole or in part, by the present invention, in which one of the embodiments of the present invention involves a method of assaying susceptibility and/or confirming diagnosis of breast fibroadenomas development in a human subject. The method essentially comprises the steps of performing a nucleic acid-based assay to analyze an isolated polynucleotide encoding at least exon 2 of MED12 gene from a sample acquired from the human subject; and regarding the human subject with greater susceptibility and/or confirming diagnosis of breast fibroadenomas development by detecting a mutation in the isolated polynucleotide. Preferably, the mutation is a splice site mutation located at position −8 of exon 2 of the MED12 gene, a missense mutation located at codon 44 of cDNA of the MED12 gene or a missense mutation located at codon 36 of cDNA of the MED12 gene.

In several preferred embodiments, the sample comprises stromal tissues which may acquire the mutation through one or more somatic events progressively acquired in the subject.

Some embodiments of the disclosed method preferably detect the missense mutation, which is located at nucleotide position 107 of codon 36 cDNA of the MED12 gene.

For a number of embodiments, the missense mutation is located at position 130 and/or 131 of codon 44 cDNA of the MED12 gene. More preferably, the missense mutation results in p.G44A, p.G44C, p.G44D, p.G44R, p.G44S, or p.G44V in a polypeptide translated from the MED12 gene.

In several preferred embodiments, the disclosed method may include additional steps of detecting at least one mutation located at PIK3CA and/or TP53 gene of the subject upon detecting a mutation in the isolated polynucleotide encoding at least exon 2 of MED12 gene; and regarding developed fibroadenoma in the subject as benign state in the absence of detectable mutation located at PIK3CA and/or TP53 gene.

### BRIEF DESCRIPTION OF THE DRAWINGS

FIG. 1 is a schematic diagram showing the distribution of MED12 exon 2 mutations in which the top panel shows regions of deletions, the middle panel and bottom respectively show nucleotide changes associated with point mutations and the corresponding codon alterations;

FIG. 2 shows results of Genomic DNA Sanger sequencing of MED12 variants in eight fresh frozen FAs and their matched whole-blood;

FIG. 3 shows results of Complementary DNA (cDNA) Sanger sequencing of MED12 variants in eight fresh frozen FAs and their matched whole-blood that variant peaks were unambiguous except for Sample002, possibly due to RNA degradation;

FIG. 4 (a) is Hematoxylin and eosin (H&E) stained section of Sample006 with the epithelial compartments marked in green and (b) shows respective Sanger sequencing results of MED12 bulk tissue, epithelial and stromal compartments, revealing that p.G44D mutations in MED12 are exclusive to the stromal compartment;

FIG. 5 (a) is a heat map showing differential activation of gene sets associated with breast cancer and estrogen signaling associated with MED12 alterations in FA and unsupervised clustering of gene sets with significantly differential activation scores as determined by GSVA, and (b) is a GSEA enrichment plot that genes are rank-ordered according to fold-change between mutant MED12 and wild-type MED12 FA samples (bottom panel), with Genes upregulated >4× in UL being indicated as black bars in the middle panel;

FIG. 6 is a GSEA enrichment plot against genes upregulated 2× in UL instead of 4×, as shown in FIG. 5b; and

FIG. 7 shows (a) cDNA sequence of MED12, as in Seq ID No.15, and (b) amino acid sequence of MED12 peptide, as in Seq ID No.16.

### DETAILED DESCRIPTION

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the description provided hereinafter. All changes that come within the meaning and range of equivalency of the claims are to be embraced within their scope.

Unless specified otherwise, the terms “comprising” and “comprise” as used herein, and grammatical variants thereof, are intended to represent “open” or “inclusive” language such that they include recited elements but also permit inclusion of additional, un-recited elements.

As used herein, the phrase “in embodiments” means in some embodiments but not necessarily in all embodiments.

As used herein, the terms “approximately” or “about”, in the context of concentrations of components, conditions, other measurement values, etc., means+/−5% of the stated value, or +/−4% of the stated value, or +/−3% of the stated value, or +/−2% of the stated value, or +/−1% of the stated value, or +/−0.5% of the stated value, or +/−0% of the stated value.

The term “polynucleotide” or “nucleic acid” as used herein designates mRNA, RNA, cRNA, cDNA or DNA. The term typically refers to oligonucleotides greater than 30 nucleotide residues in length.

The term “primer” used herein throughout the specification refers to an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. Primers can be “substantially complementary” to the sequence on the template to which it is designed to hybridize and serve as a site for the initiation of synthesis. By “substantially complementary”, it is meant that the primer is sufficiently complementary to hybridize with a target polynucleotide. Preferably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

The term “gene” as used herein may refer to a DNA sequence with functional significance. It can be a native nucleic acid sequence, or a recombinant nucleic acid sequences derived from natural source or synthetic construct. The term “gene” may also be used to refer to, for example and without limitation, a cDNA and/or an mRNA encoded by or derived from, directly or indirectly, genomic DNA sequence.

One aspect of the present disclosure refers to a method of assaying susceptibility and/or confirming diagnosis of breast fibroadenomas development in a human subject, preferably a female human subject. Essentially, the method of assaying comprises the steps of performing a nucleic acid-based assay to analyze an isolated polynucleotide encoding at least exon 2 of MED12 gene from a sample acquired from the human subject; and regarding the human subject with greater susceptibility and/or confirming diagnosis of breast fibroadenomas development by detecting a mutation in the isolated polynucleotide. The sample applicable for the disclosed method can be any biological sample having the extractable or accessible genetic materials suspected to have carrying the mutations of interested, either acquired or constitutional, and detectable in the nucleic acid-based assay. The sample can be, but not limited to, biopsy tissue or blood sample of the subject. More preferably, the sample or the biopsy tissue comprises stromal tissues, which is found by the inventors of the present disclosure being prone to adversely affected, likely owing to dysregulated extracellular matrix organization, in a significant extent by the mutations of interested located at exon 2 of the MED12 gene. The sample may be subjected to pre-treatment to isolate the preferred tissue type prior to extracting the genetic material for analysis.

Further, the polynucleotide to be reacted or analyzed in the nucleic-acid based assay can be directly or indirectly derived from the genetic materials extracted or obtained from the sample of the subject. Polynucleotides can be acquired directly from the sample source, but not limited to, by digesting or cutting the targeted gene segment utilizing restriction enzymes recognizing the specific restriction site located adjacent to the interested portion. On the other hand, the polynucleotides can be amplicons generated and duplicated from the extracted genetic materials through any known PCR or the like approaches. These amplicons are further subjected to the analysis of the nucleic acid-based assay to identify the possible mutations resulting in the occurrence of fibroadenomas.

According to several preferred embodiments, the nucleic acid-based assay can be performed to identify and/or detect the mutations comprises sequencing the polynucleotide. More specifically, the sequencing approach implementable in the present disclosure to effect the detection can be Sanger sequencing and/or ultra-deep targeted amplicon sequencing which is effective and capable of catering highly precise and reliable result in identifying the interested mutations in exon 2 of the MED12 gene. Primer pairs Seq ID No. 1 and Seq ID No. 2 listed in Table 1 below are one embodiment of the primers usable in the present disclosure to realize the sequencing process on exon 2 of the MED12 gene. The sequencing process shall provide reliable reading about the sequence of the polynucleotide that substantial outcome can be inferred thereby regarding the tested subject, at least the in the sample, whether the FA-associated mutation are carried. Furthermore, Seq ID No.3-14 are sequences of the primers pairs can be used to perform the ultra-deep targeted amplicon sequencing. The details of the Sanger sequencing and/or ultra-deep targeted amplicon sequencing utilizing the listed primer pairs are further elaborated in the examples incorporated hereafter. It is important for other skilled artisans to appreciate the fact that the disclosed method can be conducted utilizing other known sequencing equivalent or non-equivalent procedures or approaches to detect presence of the interested mutation in the analyzed polynucleotides and such modification shall not depart from the scope of the present disclosure. Other known processes implementable to identify or assist in identifying these mutations can be any one of, but not limited to, temperature gradient gel electrophoresis, capillary electrophoresis, amplification-refractory mutation system-polymerase chain reaction (ARMS-PCR), dynamic allele-specific hybridization (DASH), target capture for next generation sequencing (NGS), high-density oligonucleotide SNP arrays or Restriction fragment length polymorphism (RFLP).

TABLE 1
Primers for sequencing

| | Sequences (5′ to 3′) |
|---|---|
| Primer pairs of | |
| Sanger sequencing | |
| Seq ID No. 1 | TGTTCTACACGGAACCCTCCTC |
| Seq ID No. 2 | CTGGGCAAATGCCAATGAGAT |
| Primer pairs for | |
| ultra-deep sequencing | |
| Seq ID No. 3 | TTCTCCTGCCCTACTCTCCCAC |
| Seq ID No. 4 | CAGGCTGGTTATTGAAACCTTG |
| Seq ID No. 5 | CCCTAAGGAAAAAACAACTAAACGC |
| Seq ID No. 6 | CTGCCATGCTCATCCCCAGA |
| Seq ID No. 7 | CTTGTTCCTTCTTTTCTCCTGCC |
| Seq ID No. 8 | GTTTTACATTCAAGGCCGTCAG |
| Seq ID No. 9 | CAACTAAACGCCGCTTTCCTG |
| Seq ID No. 10 | AAGCTGACGTTCTTGGCACTGC |
| Seq ID No. 11 | GCTTTCCTGCCTCAGGATGAACT |
| Seq ID No. 12 | CCTTGGCAGGATTGAAGCTGAC |
| Seq ID No. 13 | GATGAACTGACGGCCTTGAATGTA |
| Seq ID No. 14 | CCTGGCAGAGTTGTCTCACCTTG |

Pursuant to the preferred embodiments, the disclosed method targets to analyze and/or identify multiple potential mutations residing in exon 2 the MED12 gene concurrently. One of the interesting mutations is a splice site mutation located at position −8 of exon 2 of the MED12 gene. More specifically, the splice site mutation is an intronic T>A substitution located 8 bp upstream of exon 2 of the MED12 gene in the genomic DNA. This splice site mutation results in an aberrant splice acceptor site further leading to retiontion of the last six bases of the MED12 gene intron 1 in the mRNA transcribed thereof.

Another mutation to be identified by the disclosed method, in a number of embodiments, is a missense mutation located at codon 36 of cDNA of the MED12 gene. Preferably, the missense mutation is located at position 107 of codon 36 cDNA of the MED12 gene causing a non-synonymous substitution of the encoded amino acid thereof. More preferably, the disclosed method seeks to detect presence of any mutation resulting in any one of p.L36R or p.L36P. Correspondingly, the equivalent mutations positioned on the cDNA result in the nonsynonymous substitution of p.L36R and p.L36P are respectively c.107T>G and c.107T>A. Considering degeneracy of the codon involved, other mutations may result in similar synonymous substitution of the involved amino acids, p.L36R and p.L36P, besides c.107T>G and c.107T>A.

According to other preferred embodiments, the disclosed method also aims to identify a missense mutation located at codon 44 of cDNA of the MED12 gene. Specifically, the missense mutation is located at position 130 and/or 131 of codon 44 cDNA of the MED12 gene giving rise to non-synonymous substitution of encoded amino acids such as p.G44A, p.G44C, p.G44D, p.G44R, p.G44S, or p.G44V in a polypeptide translated from the MED12 gene.

It is important to note that inventors of the present disclosure found that the aforesaid mutations may subsequently upregulate or activate other genes associated with extracellular matrix organization, estrogen signaling, and TGFβ and Wnt signaling. Up-regulation or uncontrolled activation of these genes or gene products shall hence promote development of FA in the subject.

In several preferred embodiments, the disclosed method may include additional steps of detecting at least one mutation located at PIK3CA and/or TP53 gene of the subject upon detecting a mutation in the isolated polynucleotide encoding at least exon 2 of MED12 gene; and regarding developed fibroadenoma in the subject as benign state in the absence of detectable mutation located at PIK3CA and/or TP53 gene.

Another aspect of the present disclosure may include use of Seq. ID No. 1 and 2 in the preparation of a platform for nucleic-acid based assay for assaying susceptibility and/or confirming diagnosis of breast fibroadenomas development in a female human subject.

Likewise, in further aspect, the present disclosure may include use of Seq. ID No. 3 and 4, Seq. ID No. 5 and 6, Seq. ID No. 7 and 8, Seq. ID No. 9 and 10, Seq. ID No. 11 and 12, and/or Seq. ID No. 13 and 14 in preparation of a platform for nucleic-acid based assay for assaying susceptibility and/or confirming diagnosis of breast fibroadenomas development in a female human subject. Preferably, the use of Seq. ID No. 3 and 4, Seq. ID No. 5 and 6, Seq. ID No. 7 and 8, Seq. ID No. 9 and 10, Seq. ID No. 11 and 12, and/or Seq. ID No. 13 and 14 in the mentioned platform facilitates or materializes identification of a missense mutation located at codon 44 of cDNA of MED12 gene or a missense mutation located at codon 36 of cDNA of MED12 gene. Presence of at least one of these mutations in the breast tissue, more preferably stromal cells of the breast tissue, has been shown to associate with greater risk in developing FA in relation to those with the wild type allele by the present disclosure.

The following example is intended to further illustrate the invention, without any intent for the invention to be limited to the specific embodiments described therein.

### Example 1

A total of 98 fibroadenoma tumors were included in this study, of which 12 were from fresh frozen tumors and a further 86 from archival FFPE (formalin-fixed paraffin-embedded) samples. Tumors and whole-blood were obtained from patients undergoing surgical excision of fibroadenoma or from the SingHealth Tissue Repository, with signed informed consent. Archival samples were obtained from the Department of Pathology of Singapore General Hospital. Clinicopathological information for subjects (age and tumor size) was reviewed retrospectively.

Genomic DNA (gDNA) from fresh frozen tissue was extracted and purified using the Qiagen Blood and Cell Culture DNA kit. In the case of FFPE samples, the Qiagen FFPE DNA kit was used on freshly sectioned FFPE tissue. Genomic DNA yield and quality were determined using Picogreen™ fluorometric analysis as well as visual inspection of agarose gel electrophoresis images.

### Example 2

Native genomic DNA was fragmented with the Covaris™ S2 (Covaris) system using recommended settings. Sequencing adaptor ligation was performed using the Truseq Paired-End Genomic DNA kit (Illumina). For enrichment of coding sequences, the present disclosure used the SureSelectXT™ Human All Exon v3 (50 Mb) kit (Agilent Technologies) according to manufacturer's recommended protocol. Exome-enriched libraries were then sequenced on Illumina's HiSeq 2000 sequencing platform to generate 76 bp paired-end reads. Bioinformatics analysis, comprising of sequence alignment, variant calling and identification of candidate somatic variants was performed as described in previous work²⁸. For point mutations, at least 10 variant reads in the tumor and a total read depth of 10 in the normal sample was required. In the case of Indels, a support of at least 20 variant reads amounting to at least 10% of total reads was required. Indels overlapping simple repeat regions were also discarded. All remaining candidate variants were visually inspected in a genome browser to identify missed probable germline variants or those in regions of anomalous alignment. The variant calling pipeline missed the p.Glu33_Asp34insProGln aberrant splice site variant in Sample004 as it was not in the exome capture kit manufacturer's target region file. It was later identified from a systematic visual inspection of MED12 in a genome browser as it was the only gene recurrently mutated in multiple samples. All candidate somatic variants were confirmed by Sanger sequencing.

The present disclosure used the following PCR primer pair to identify mutations in MED12 exon 2; forward primer: TGTTCTACACGGAACCCTCCTC, reverse primer: CTGGGCAAATGCCAATGAGAT, Tm: 54.6 C, 56.3 C, product length: 373 bp. PCR amplification was conducted using neat DNA and Platinum™ Taq Polymerase (Life Technologies). PCR cycling regime included one cycle at 95° C. for 10 min, 35 cycles at 95° C. for 30 s, 58° C. for 30 s and 72° C. for 1 min, and one cycle at 72° C. for 10 min. BigDye Terminator v.3.1 kit (Applied Biosystems) was used for bi-directional sequencing on generated PCR amplicons and products were fractionated employing ABI PRISM 3730 Genetic Analyzer (Applied Biosystems). Sequencing traces were aligned to reference sequences using Lasergene 10.1 (DNASTAR) and were visually analyzed.

### Example 3

For sensitive detection of low-frequency variants in MED12 exon 2, the present disclosure further used ultra-deep targeted amplicon sequencing. Six PCR amplicons were designed and tiled across exon 2 of MED12 using primers pairs listed in Table 2 below.

TABLE 2
PCR amplicon sequencing primers used in ultra-
deep targeted amplicon sequencing of MED12 exon 2

| | | | | | PCR |
|---|---|---|---|---|---|
| | | | Primer | | product |
| | | | sequence | Melting | size |
| Gene | Region | Primer Name | (5′->3′) | Point | (bp) |
| MED12 | Exon2 | MD12-ex2-2F | TTCTCCTGC | 56.2 | 125 |
| | | | CCTACTCTC | | |
| | | | CCAC | | |
| | | MD12-ex2-2R | CAGGCTGGT | 53.2 | |
| | | | TATTGAAAC | | |
| | | | CTTG | | |
| | | MD12-ex2-3F | CCCTAAGGA | 55.9 | 115 |
| | | | AAAAACAAC | | |
| | | | TAAACGC | | |
| | | MD12-ex2-3R | CTGCCATGC | 58.1 | |
| | | | TCATCCCCA | | |
| | | | GA | | |
| | | MD12-ex2-1F | CTTGTTCCT | 55.3 | 117 |
| | | | TCTTTTCTC | | |
| | | | CTGCC | | |
| | | MD12-ex2-1R | GTTTTACAT | 53.2 | |
| | | | TCAAGGCCG | | |
| | | | TCAG | | |
| | | MD12-ex2-4F | CAACTAAAC | 56.4 | 119 |
| | | | GCCGCTTTC | | |
| | | | CTG | | |
| | | MD12-ex2-4R | AAGCTGACG | 58.2 | |
| | | | TTCTTGGCA | | |
| | | | CTGC | | |
| | | MD12-ex2-5F | GCTTTCCTG | 57.6 | 121 |
| | | | CCTCAGGAT | | |
| | | | GAACT | | |
| | | MD12-ex2-5R | CCTTGGCAG | 57.5 | |
| | | | GATTGAAGC | | |
| | | | TGAC | | |
| | | MD12-ex2-6F | GATGAACTG | 57 | 124 |
| | | | ACGGCCTTG | | |
| | | | AATGTA | | |
| | | MD12-ex2-6R | CCTGGCAGA | 57.6 | |
| | | | GTTGTCTCA | | |
| | | | CCTTG | | |

The present disclosure then used Fluidigm's Access Array System to generate and pool the amplicons according to manufacturer's instructions. For each sample, 50 ng of genomic DNA was used as template. Sequencing library preparation of the pooled amplicons was performed using the TruSeq HT DNA Sample Preparation Kit (Illumina) according to manufacturer's instructions. Sequencing was performed on the Illumina MiSeq next-generation sequencing platform for 150 cycles using the MiSeq Reagent kit v3.

Bioinformatics analysis of sequencing reads was performed as follows. Briefly, undetermined (‘N’) base calls at the ends of reads were trimmed. Following this, the 5′ end of each read was trimmed by 25 bases to eliminate the possibility of primer inclusion. The Burrows-Wheeler Alignment²⁶ (BWA) tool (0.6.2) was used to align the resulting reads to the reference human genome (hg19). For more sensitive detection of insertions and deletions (indels), the present disclosure also ran a separate alignment process using modified settings (o=2, e=30, d=30, O=0, E=0, L=0). Indels were identified through manual inspection, whereas automated detection of point mutations was performed using the samtools²⁷ (0.1.18) mpileup tool. Variant calls were restricted to regions covered by amplicons generated from primers pairs provided in the Table 1. Variant allele frequencies were calculated for each position in the targeted region, and those that exceeded a threshold of 5% were considered candidate variants. In order to minimize the possibility of PCR-induced artifacts, variants were only considered valid if present in at least two amplicons. Candidate variants had at least 21,620 sequencing reads overlapping them, with an average coverage of 184,526 reads.

To ascertain the sensitivity of our assay, positive control samples containing spiked-in validated mutant MED12 at allele frequencies (15%, 10%, 5%, 3%) were generated via serial dilution. The present disclosure accurately detected variants in positive control samples at allele frequencies down to 3%. The present disclosure also calculated alternate (nonreference) allele frequencies across all positions in our target region in order to estimate the likelihood of error from sequencing and alignment artifacts. The mean alternate allele frequency was 0.281% with a standard deviation of 1.09%. Thus, our detection threshold of 5% exceeds four standard deviations from the estimated background error rate.

To identify genes with recurrent somatic mutations across multiple samples, the present disclosure first sequenced the exomes of eight fresh frozen FA tumors together with matched whole-blood to a mean coverage of 124×, with an average of 87% of bases covered by at least 20 reads in each sample.

TABLE 3
Summary of whole-exome sequencing of FA tumors and matched normal tissue (whole-
blood). Highlighted samples contain somatic MED12 exon 2 mutations.

| | | | | Reads | Ave. | Targeted | Targeted | |
|---|---|---|---|---|---|---|---|---|
| | | | Bases in | Mapped to | Depth Per | Bases with | Bases with | Candidate |
| | | Sample | Target | Target | Targeted | Depth at | Depth at | somatic |
| No. | Sample | Type | Region | Region* | Base | Least 1X (%) | Least 20X (%) | mutations |
| 1 | Sample002N | Normal | 51,860,012 | 117,269,831 | 130 | 95.6 | 88 | 1 |
| | Sample002T | Tumor | 51,860,012 | 164,371,773 | 184 | 95.9 | 90 | |
| 2 | Sample004N | Normal | 51,860,012 | 114,347,376 | 127 | 95.6 | 88 | 19 |
| | Sample004T | Tumor | 51,860,012 | 114,347,376 | 106 | 95.5 | 86 | |
| 3 | Sample006N | Normal | 51,860,012 | 98,767,446 | 111 | 95.4 | 86 | 7 |
| | Sample006T | Tumor | 51,860,012 | 161,158,144 | 155 | 95.8 | 90 | |
| 4 | Sample007N | Normal | 51,860,012 | 116,511,803 | 130 | 95.4 | 88 | 7 |
| | Sample007T | Tumor | 51,860,012 | 113,165,060 | 126 | 95.5 | 88 | |
| 5 | Sample009N | Normal | 51,860,012 | 83,736,104 | 93 | 95.5 | 86 | 7 |
| | Sample009T | Tumor | 51,860,012 | 114,378,362 | 128 | 95.6 | 88 | |
| 6 | Sample010N | Normal | 51,860,012 | 119,910,977 | 136 | 95.5 | 87 | 4 |
| | Sample010T | Tumor | 51,860,012 | 98,563,020 | 110 | 95.4 | 86 | |
| 7 | Sample011N | Normal | 51,860,012 | 112,204,764 | 125 | 95.5 | 88 | 1 |
| | Sample011T | Tumor | 51,860,012 | 94,612,966 | 105 | 95.3 | 86 | |
| 8 | Sample012N | Normal | 51,860,012 | 84,410,000 | 95 | 95.3 | 85 | 5 |
| | Sample012T | Tumor | 51,860,012 | 114,280,813 | 128 | 95.5 | 88 | |

| | | | | | |
|---|---|---|---|---|---|
| Average | 113,877,238 | 124 | 96 | 87 | 6 |

Consistent FA being a benign tumor, samples had an average of only seven somatic mutations. Almost all genes were found to be mutated only once. These included tumor suppressors such as NF1 and RB1. The only gene that was recurrently mutated was MED12 (mediator complex subunit 12), which is a member of the Mediator Complex, a multiprotein complex that is widely involved in transcriptional regulation of gene expression. Four out of the eight FA samples sequenced (50%) contained somatic mutations in exon 2 of MED12 as presented in Table 4.

TABLE 4
List of candidate somatic mutations identified from whole-exome sequencing of eight FAs.

| | | | | | | Amino | |
|---|---|---|---|---|---|---|---|
| | Gene | | | Nucleotide | Nucleotide | acid | Mutation |
| No | Symbol | Sample | Transcript ID | (genomic) | (cDNA) | (protein) | type |
| 1 | MED12 | Sample002 | CCDS43970.1 | g.chrX: 70339254 | c.131 G > A | p.G44D | Missense |
| | | | | G > A | | | |
| 2 | MED12 | Sample006 | CCDS43970.1 | g.chrX: 70339254 | c.131 G > A | p.G44D | Missense |
| | | | | G > A | | | |
| 3 | MED12 | Sample007 | CCDS43970.1 | g.chrX: 70339254 | c.131 G > A | p.G44D | Missense |
| | | | | G > A | | | |
| 4 | MED12 | Sample004 | CCDS43970.1 | g.chrX: 70339215 | c.100−8 T > A | Splice | Splice |
| | | | | T > A | | site | site |
| 5 | ANK2 | Sample004 | CCDS3702.1 | g.chr4: 114277287 | c.84+29032 | p.V2505L | Missense |
| | | | | G > T | G > T | | |
| 6 | C1orf173 | Sample007 | CCDS30755.1 | g.chr1: 75037715 | c.3679 G > T | p.V1227L | Missense |
| | | | | G > T | | | |
| 7 | C22orf23 | Sample004 | CCDS13962.1 | g.chr22: 38340198 | c.638 | Frameshift | Indel |
| | | | | delCCTT | delCCTT | | |
| 8 | CC2D1A | Sample006 | CCDS42512.1 | g.chr19: 14034557 | c.1873 | Frameshift | Indel |
| | | | | delCT | delCT | | |
| 9 | CHD6 | Sample012 | CCDS13317.1 | g.chr20: 40033303 | c.8078 G > A | p.P2693L | Missense |
| | | | | G > A | | | |
| 10 | CKAP5 | Sample012 | CCDS31477.1 | g.chr11: 46799825 | c.2612 A > G | p.D871G | Missense |
| | | | | A > G | | | |
| 11 | CREBBP | Sample004 | CCDS45399.1 | g.chr16: 3786805 | c.4292 | Frameshift | Indel |
| | | | | delCT | delCT | | |
| 12 | DNAH11 | Sample009 | NM_001277115 | g.chr7: 21781777 | c.8178 T > A | p.V2716D | Missense |
| | | | | T > A | | | |
| 13 | FGB | Sample004 | CCDS3786.1 | g.chr4: 155487155 | c.306+4 | Splice | Splice |
| | | | | G > T | G > T | site | site |
| 14 | FRMD4A | Sample004 | CCDS7101.1 | g.chr10: 13804618 | c.441+6 | Splice | Splice |
| | | | | T > A | T > A | site | site |
| 15 | GRIN3B | Sample012 | CCDS32861.1 | g.chr19: 1003331 | c.629 C > T | p.T210M | Missense |
| | | | | C > T | | | |
| 16 | ISL1 | Sample009 | CCDS43314.1 | g.chr5: 50685533 | c.532 C > T | p.P178S | Missense |
| | | | | C > T | | | |
| 17 | IST1 | Sample004 | CCDS10905.1 | g.chr16: 71956504 | c.680 C > T | p.T227M | Missense |
| | | | | C > T | | | |
| 18 | KCNG4 | Sample006 | CCDS10945.1 | g.chr16: 84255957 | c.1426 C > T | p.R476C | Missense |
| | | | | C > T | | | |
| 19 | KIAA1211L | Sample004 | CCDS42720.1 | g.chr2: 99454665 | c.156 C > A | p.S52R | Missense |
| | | | | C > A | | | |
| 20 | KRTAP1-3 | Sample006 | CCDS42323.1 | g.chr17: 39190785 | c.289 delCT | Frameshift | Indel |
| | | | | delCT | | | |
| 21 | LAMB4 | Sample010 | CCDS34732.1 | g.chr7: 107735743 | c.1400 C > G | p.T467S | Missense |
| | | | | C > G | | | |
| 22 | LPA | Sample006 | CCDS43523.1 | g.chr6: 160998309 | c. 4289+6078 | p.P1497L | Missense |
| | | | | C > T | C > T | | |
| 23 | LRRC10 | Sample012 | CCDS31856.1 | g.chr12: 70004273 | c.346 G > A | p.E116K | Missense |
| | | | | G > A | | | |
| 24 | LRRC42 | Sample012 | CCDS585.1 | g.chr1: 54432042 | c.1001 C > G | p.A334G | Missense |
| | | | | C > G | | | |
| 25 | LRRTM3 | Sample011 | CCDS7270.1 | g.chr10: 68686900 | c.226 delT | Frameshift | Indel |
| | | | | delT | | | |
| 26 | MAGEE1 | Sample009 | CCDS14433.1 | g.chrX: 75650516 | c.2193 T > G | p.Y731X | Missense |
| | | | | T > G | | | |
| 27 | MAPT | Sample006 | CCDS45715.1 | g.chr17: 44055797 | c.364 G > A | p.V122M | Missense |
| | | | | G > A | | | |
| 28 | MYO9A | Sample007 | CCDS10239.1 | g.chr15: 72170501 | c.5811 G > A | p.M1937I | Missense |
| | | | | G > A | | | |
| 29 | NF1 | Sample009 | CCDS42292.1 | g.chr17: 29560073 | c.3550 A > T | p.T1184S | Missense |
| | | | | A > T | | | |
| 30 | NODAL | Sample004 | CCDS7304.1 | g.chr10: 72195115 | C.818 C > T | p.A273V | Missense |
| | | | | C > T | | | |
| 31 | NOTCH2 | Sample004 | CCDS908.1 | g.chr1: 120491681 | c.2548 | Frameshift | Indel |
| | | | | delTT | delTT | | |
| 32 | NUMA1 | Sample004 | CCDS31633.1 | g.chr11: 71724080 | c.4469 G > T | p.R1490L | Missense |
| | | | | G > T | | | |
| 33 | PCLO | Sample010 | CCDS47630.1 | g.chr7: 82580690 | c.9214 C > T | p.P3072S | Missense |
| | | | | C > T | | | |
| 34 | PGAP1 | Sample004 | CCDS2318.1 | g.chr2: 197791238 | c.103 | In-frame | Indel |
| | | | | delCTC | delCTC | | |
| 35 | POM121L12 | Sample004 | CCDS43584.1 | g.chr7: 53103758 | c.394 C > T | p.R132W | Missense |
| | | | | C > T | | | |
| 36 | POTEA | Sample007 | NM_001002920 | g.chr8: 43211931 | c.1295 G > T | p.A464S | Missense |
| | | | | G > T | | | |
| 37 | PRAF2 | Sample009 | CCDS14317.1 | g.chrX: 48929554 | c.511 G > C | p.G171R | Missense |
| | | | | G > C | | | |
| 38 | PSME4 | Sample009 | CCDS33197.2 | g.chr2: 54158971 | c.1316+1 | Splice | Splice |
| | | | | G > A | G > A | site | site |
| 39 | RARA | Sample007 | CCDS11366.1 | g.chr17: 38510626 | c.880 C > T | p.R294W | Missense |
| | | | | C > T | | | |
| 40 | RB1 | Sample004 | CCDS31973.1 | g.chr13: 48881465 | c.187 G > T | p.K63X | Missense |
| | | | | G > T | | | |
| 41 | RB1 | Sample004 | CCDS31973.1 | g.chr13: 48937094 | c.861+1 | Splice | Splice |
| | | | | A > T | A > T | site | site |
| 42 | ROS1 | Sample004 | CCDS5116.1 | g.chr6: 117609731 | c.6968 A > T | p.Y2323F | Missense |
| | | | | A > T | | | |
| 43 | SAAL1 | Sample006 | CCDS31439.1 | g.chr11: 18112008 | c.446 A > G | p.D149G | Missense |
| | | | | A > G | | | |
| 44 | SCN10A | Sample004 | CCDS33736.1 | g.chr3: 38783906 | c.1982 T > C | p.L661P | Missense |
| | | | | T > C | | | |
| 45 | SEMA4F | Sample007 | CCDS1955.1 | g.chr2: 74902152 | c.1139 G > T | p.R380I | Missense |
| | | | | G > T | | | |
| 46 | SHROOM4 | Sample007 | CCDS35277.1 | g.chrX: 50350882 | c.3260 C > T | p.T1087I | Missense |
| | | | | C > T | | | |
| 47 | SIAH3 | Sample009 | CCDS41883.1 | g.chr13: 46357894 | c.434 C > T | p.A145V | Missense |
| | | | | C > T | | | |
| 48 | SYNE4 | Sample004 | NM_001039876 | g.chr19: 36494181 | c.1362 C > A | p.T365N | Missense |
| | | | | C > A | | | |
| 49 | TNFAIP3 | Sample010 | CCDS5187.1 | g.chr6: 138196931 | c.593 T > C | p.V198A | Missense |
| | | | | T > C | | | |
| 50 | TRPM1 | Sample010 | CCDS58347.1 | g.chr15: 31323296 | c.3068 G > A | p.R1023H | Missense |
| | | | | G > A | | | |

### Example 4

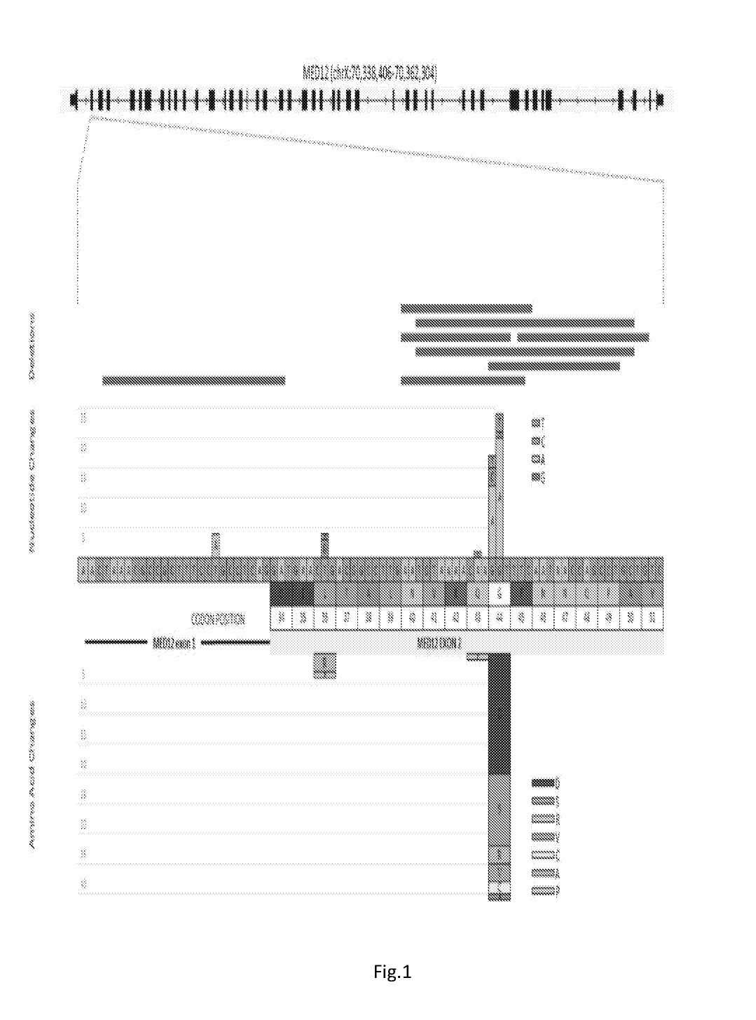

In order to further ascertain the prevalence of MED12 exon 2 mutations in FA, the present disclosure performed ultra-deep targeted amplicon sequencing of MED12 exon 2 in 90 additional FA samples (4 fresh frozen tissue samples and 86 archival samples). This confirmed a strikingly high MED12 exon 2 mutation frequency in FA of 59%. Frequency of the various detected mutation is summarized in Table 5 and FIG. 1.

TABLE 5
A tabular summary of MED12 exon 2 mutations in Fa in comparison with
corresponding mutation frequencies in UL are indicated where applicable (FA =
fibroadenoma, UL = uterine leiomyoma, ins = insertion, del = deletion, fs = frameshift).

| | | | # mutated out of | # mutated out of |
|---|---|---|---|---|
| | | | 98 samples in | 225 samples in |
| Type | cDNA | Protein | FA (%) | UL¹³ (%) |
| Misssense | c.131G > C | p.G44A | 1 (1.1) | 11 (5.0) |
| | c.130G > T | p.G44C | 2 (2.2) | 7 (3.1) |
| | c.131G > A | p.G44D | 20 (20.4) | 47 (20.9) |
| | c.130G > C | p.G44R | 3 (3.3) | 16 (7.1) |
| | c.130G > A | p.G44S | 12 (13.3) | 17 (7.6) |
| | c.131G > T | p.G44V | 3 (3.3) | 12 (5.3) |
| | c.128A > C | p.Q43P | 1 (1.1) | 3 (1.3) |
| | c.107T > G | p.L36R | 3 (3.3) | 11 (5.0) |
| | c.107T > A | p.L36P | 1 (1.0) | 0 (0.0) |
| Splice Site | Exon2 (−8 T > A) | p.E33_D34insPQ | 4 (4.1) | 10 (4.4) |
| Deletions | intronic −23bp | p.D34fs | 1 (1.1) | 0 (0.0) |
| | c.100_101del2 | | | |
| | c.134_151del18 | p.F45_V51 > F | 1 (1.1) | 0 (0.0) |
| | c.130_147del18 | p.G44_P49 | 1 (1.1) | 0 (0.0) |
| | c.120_149del30 | p.N40_A50 > N | 2 (2.2) | 0 (0.0) |
| | c.118_132del15 | p.N40_G44 | 1 (1.1) | 0 (0.0) |
| | c.118_135del18 | p.N40_F45 | 2 (2.2) | 0 (0.0) |

| | | |
|---|---|---|
| Total | 58 (59.2) | — |

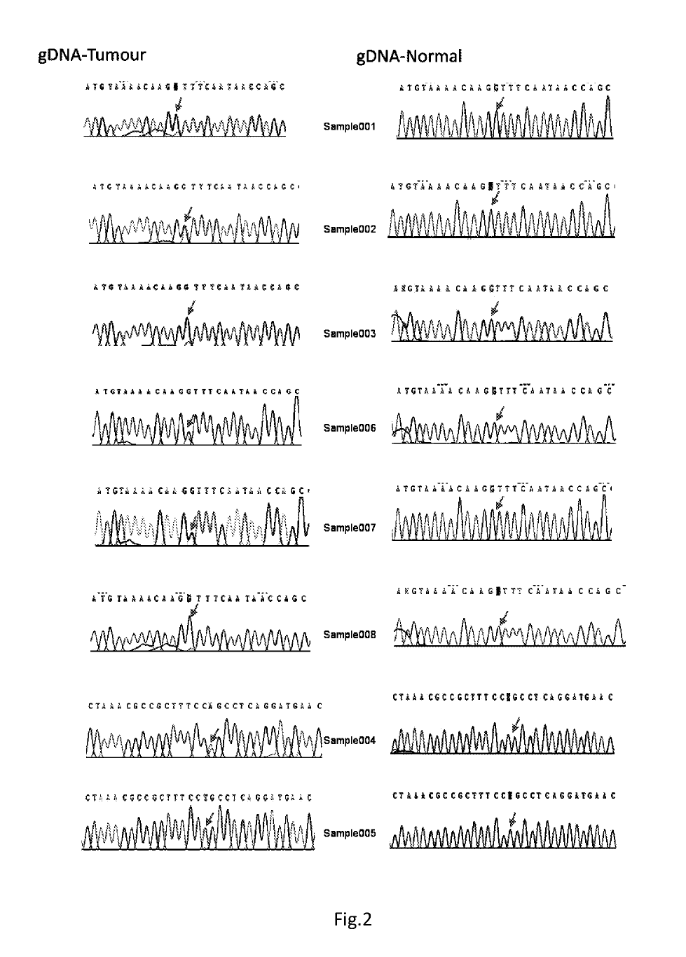

Out of the 98 FA samples sequenced, 41 (42%) had point mutations in codon 44 (20 p.G44D, 12 p.G44S, 3 p.G44R, 3 p.G44V, 2 p.G44C, 1 p.G44A). A single point mutation (1.1%) was also found in codon 43 (p.Q43P) and four (4.1%) in codon 36 (3 p.L36R, 1 p.L36P). Additionally, seven (7.8%) samples were found to have insertions or deletions that were expected to preserve the reading frame, and one (1.1%) further sample harbored a frameshift deletion. The present disclosure also identified four samples with an intronic T>A substitution 8 bp upstream of exon 2 that resulted in an aberrant splice acceptor site, causing the last six bases of intron 1 to be retained¹³. Several lines of evidence indicate the MED12 exon 2 mutations are somatic. The present disclosure performed Sanger sequencing on eight MED12 mutant fresh-frozen samples with available whole-blood and confirmed that all eight mutations were somatic as indicated in FIG. 2. All but one point mutations and 25% ( 2/8) of deletions detected in our archival samples for which there was no matched whole-blood were found to have COSMIC¹⁴ (Catalog of Somatic Mutations in Cancer) entries with reference to the Table 6, none were classified as germline variants in dbSNP¹⁵ and the 1000 Genomes Project¹⁶, and an examination of our in-house database of germline variants from a predominantly East Asian cohort of 470 subjects revealed no variants in MED12 exon 2.

TABLE 6
Mutations detected in ultra-deep targeted amplicon sequencing of MED12 exon 2 in 98 FA samples.

| | | | | Variant | | | | |
|---|---|---|---|---|---|---|---|---|
| | | | | allele | | | | |
| | Sample | Total | Variant | Frequency | Amino acid | cDNA | | |
| No. | ID | Reads | Reads | (%) | change | change | COSMIC | Tissue type |
| 19 | Sample035 | 130736 | 6222 | 5 | p.G44V | c.131G > T | COSM131597 | FFPE |
| 20 | Sample036 | 80052 | 8714 | 11 | p.G44R | c.130G > C | COSM131592 | FFPE |
| 21 | Sample037 | 273272 | 22424 | 8.21 | p.N40_A50 > N | c.120_149 | | FFPE |
| | | | | | | del30 | | |
| 22 | Sample038 | 159978 | 11466 | 7.17 | p.G44_P49 | c.130_147 | | FFPE |
| | | | | | | del18 | | |
| 23 | Sample039 | 369350 | 63858 | 17.29 | p.G44D | c.131G > A | COSM131596 | FFPE |
| 24 | Sample041 | 348710 | 69038 | 19.8 | p.G44S | c.130G > A | COSM131594 | FFPE |
| 25 | Sample042 | 102994 | 7788 | 7.56 | p.D34fs | intronic - | COSM1235330 | FFPE |
| | | | | | | 23bp | | |
| | | | | | | c.100_101 | | |
| | | | | | | del2 | | |
| 26 | Sample044 | 106860 | 10694 | 10.01 | p.G44D | c.131G > A | COSM131596 | FFPE |
| 27 | Sample045 | 509920 | 142790 | 28 | p.G44S | c.130G > A | COSM131594 | FFPE |
| 28 | Sample046 | 202270 | 36546 | 18.07 | p.G44D | c.131G > A | COSM131596 | FFPE |
| 29 | Sample047 | 146698 | 19732 | 13.45 | p.G44R | c.130G > C | COSM131592 | FFPE |
| 30 | Sample048 | 176580 | 32634 | 18.48 | p.G44S | c.130G > A | COSM131594 | FFPE |
| 31 | Sample049 | 193484 | 21046 | 10.88 | p.G44D | c.131G > A | COSM131596 | FFPE |
| 32 | Sample050 | 108602 | 17060 | 15.7 | p.G44V | c.131G > T | COSM131597 | FFPE |
| 33 | Sample051 | 243910 | 29634 | 12.15 | p.G44D | c.131G > A | COSM131596 | FFPE |
| 34 | Sample053 | 87288 | 13832 | 15.85 | p.G44D | c.131G > A | COSM131596 | FFPE |
| 35 | Sample054 | 289626 | 69788 | 24.1 | p.G44R | c.130G > C | COSM131592 | FFPE |
| 36 | Sample055 | 142914 | 16800 | 11.76 | p.L36R | c.107T > G | COSM131590 | FFPE |
| 37 | Sample056 | 292544 | 34640 | 11.8 | p.G44A | c.131G > C | COSM131595 | FFPE |
| 38 | Sample057 | 246888 | 52504 | 21.27 | p.G44C | c.130G > T | COSM131593 | FFPE |
| 39 | Sample058 | 82052 | 11428 | 13.93 | p.G44D | c.131G > A | COSM131596 | FFPE |
| 40 | Sample060 | 189936 | 21498 | 11.32 | p.F45_V51 > F | c.134_151 | | FFPE |
| | | | | | | del18 | | |
| 41 | Sample065 | 72026 | 9184 | 12.75 | p.G44D | c.131G > A | COSM131596 | FFPE |
| 42 | Sample066 | 23772 | 3504 | 14.74 | p.G44D | c.131G > A | COSM131596 | FFPE |
| 43 | Sample067 | 207170 | 35934 | 17.35 | p.G44D | c.131G > A | COSM131596 | FFPE |
| 44 | Sample068 | 21620 | 1274 | 5.89 | p.G44D | c.131G > A | COSM131596 | FFPE |
| 45 | Sample069 | 26076 | 2810 | 10.78 | p.G44D | c.131G > A | COSM131596 | FFPE |
| 46 | Sample070 | 42560 | 4516 | 10.61 | p.G44S | c.130G > A | COSM131594 | FFPE |
| 47 | Sample074 | 54226 | 7328 | 13.51 | p.G44S | c.130G > A | COSM131594 | FFPE |
| 48 | Sample077 | 268636 | 62138 | 23.13 | p.G44S | c.130G > A | COSM131594 | FFPE |
| 49 | Sample078 | 42170 | 5700 | 13.5 | p.G44S | c.130G > A | COSM131594 | FFPE |
| 50 | Sample080 | 29112 | 2618 | 8 | p.G44S | c.130G > A | COSM131594 | FFPE |
| 51 | Sample085 | 60604 | 4474 | 7.38 | p.G44S | c.130G > A | COSM131594 | FFPE |
| 52 | Sample086 | 81188 | 6800 | 8.38 | p.G44S | c.130G > A | COSM131594 | FFPE |
| 53 | Sample087 | 101244 | 7140 | 7.05 | p.N40_F45 | c.118_135 | | FFPE |
| | | | | | | del18 | | |
| 54 | Sample090 | 100526 | 114548 | 11.39 | p.G44C | c.130G > T | COSM131593 | FFPE |
| 55 | Sample091 | 43602 | 8344 | 19.14 | p.L36P | c.107T > C | | FFPE |
| 56 | Sample092 | 25034 | 3034 | 12.12 | p.E33_D34 | Exon2 | COSM131618 | FFPE |
| | | | | | insPQ | (−8 T > A) | | |
| 57 | Sample095 | 42452 | 7530 | 17.74 | p.E33_D34 | Exon2 | COSM131618 | FFPE |
| | | | | | insPQ | (−8 T > A) | | |
| 58 | Sample096 | 89982 | 19106 | 21.23 | p.G44D | c.131G > A | COSM131596 | FFPE |
| — | 288_PC3 | 49790 | 1730 | 3.59 | p.G44S | c.130G > A | COSM131594 | Spike-in |
| | | | | | | | | Control |
| — | 287_PC5 | 222990 | 13576 | 6.1 | p.G44S | c.130G > A | COSM131594 | Spike-in |
| | | | | | | | | Control |
| — | 286_PC10 | 167234 | 18412 | 11.01 | p.G44S | c.130G > A | COSM131594 | Spike-in |
| | | | | | | | | Control |
| — | 285_PC15 | 185356 | 28162 | 15.2 | p.G44S | c.130G > A | COSM131594 | Spike-in |
| | | | | | | | | Control |

### Example 5

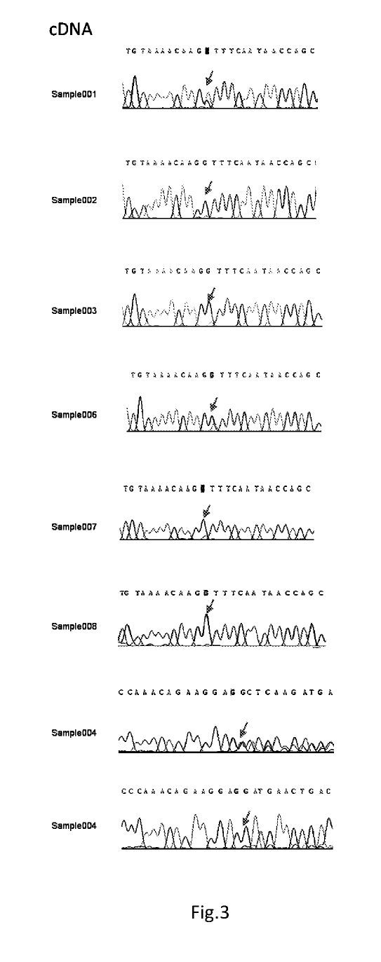

The MED12 gene lies on chromosome X, and in females, one copy is normally silenced by epigenetic inactivation¹⁷. To confirm that mutant MED12 transcripts are expressed, and are not suppressed by X-inactivation, the present disclosure performed Sanger sequencing on complementary DNA (cDNA) generated by reverse-transcribing messenger RNA (mRNA) from eight fresh frozen samples that were determined to harbor MED12 exon 2 mutations by targeted amplicon sequencing. Particularly, the present disclosure sequenced the cDNA of seven MED12-mutant samples with available fresh frozen tissue. The present disclosure converted 100 ng of RNA to cDNA with SuperScript III First-Strand Synthesis SuperMix from Invitrogen according to manufacturer's recommended protocol. The present disclosure performed PCR and sequenced the MED12 region between exon 1 and 3 with primers from Mäkinen et al¹³; forward primer: CTTCGGGATCTTGAGCTACG, reverse primer: GATCTTGGCAGGATTGAAGC, product length: 199 bp. PCR amplification, sequencing and fractionation was performed as described above for Sanger sequencing of genomic DNA. The present disclosure were able to unambiguously identify the correct MED12 mutations in the cDNA of all but one sample, as illustrated in FIG. 3, indicating that mutant MED12 is indeed transcribed.

### Example 6

Due to the biphasic nature of FA and relatively low variant allele frequencies observed in MED12 mutations (14.1%), it was suspected that MED12 mutations may be present in either the epithelial or stromal compartments. To confirm this, the present disclosure performed LCM (laser capture microdissection) on one sample (Sample006) and Sanger sequenced the individual compartments.

Briefly, fresh frozen tissue from Sample006 was embedded in Optimal Cutting Temperature (OCT) compound (Tissue-Tek, Sakura Finetek), and sections (8 μm thick) were cut in a Microtome-cryostat (Leica), mounted onto Arcturus® PEN membrane glass slides (Life Technologies), and then stored at −80° C. till required. Slides were dehydrated & stained with Arcturus® Histogene® following manufacturer's recommendations. The stained slide was loaded onto the laser capture microscope stage (ArcturusXT™ Laser Capture Microdissection (LCM) System). A Capsure™ Macro LCM cap (Life Technologies) was then placed automatically over the chosen area of the tissue. Once the cells of interest that were highlighted by the software were verified by the user, the machine automatically dissected out the highlighted cells of interest using a near infrared laser or UV pulse that transferred them onto the Capsure™ Macro LCM Cap.

The DNA was extracted directly from LCM caps using Qiagen FFPE DNA Tissue kit following manufacturer's protocol with the following modifications. Each sample cap was incubated with the lysis buffer (ATL & Proteinase K) in a 500 μl microcentrifuge at 60° C. for 5 hrs & enzyme deactivation at 90° C. for 10 minutes. The eluted DNA was used directly for PCR & BigDye® sequencing.

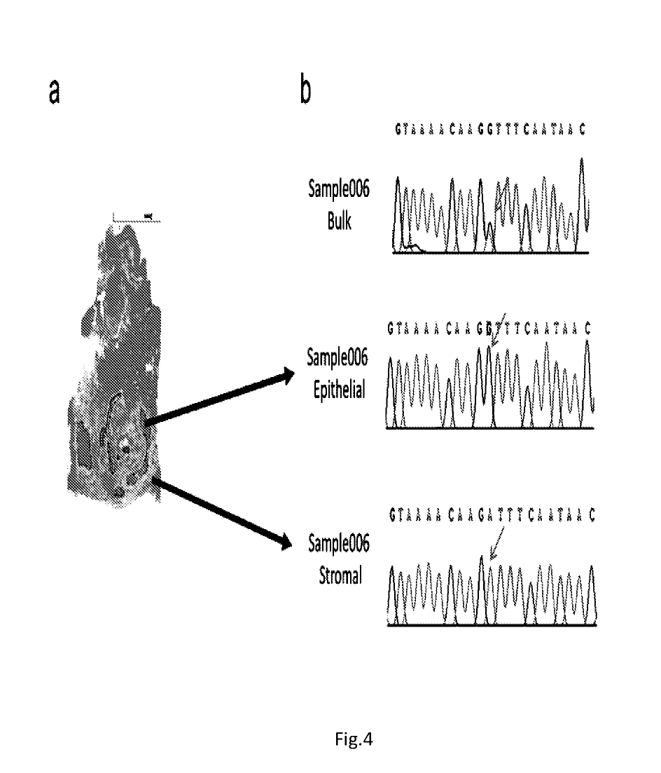

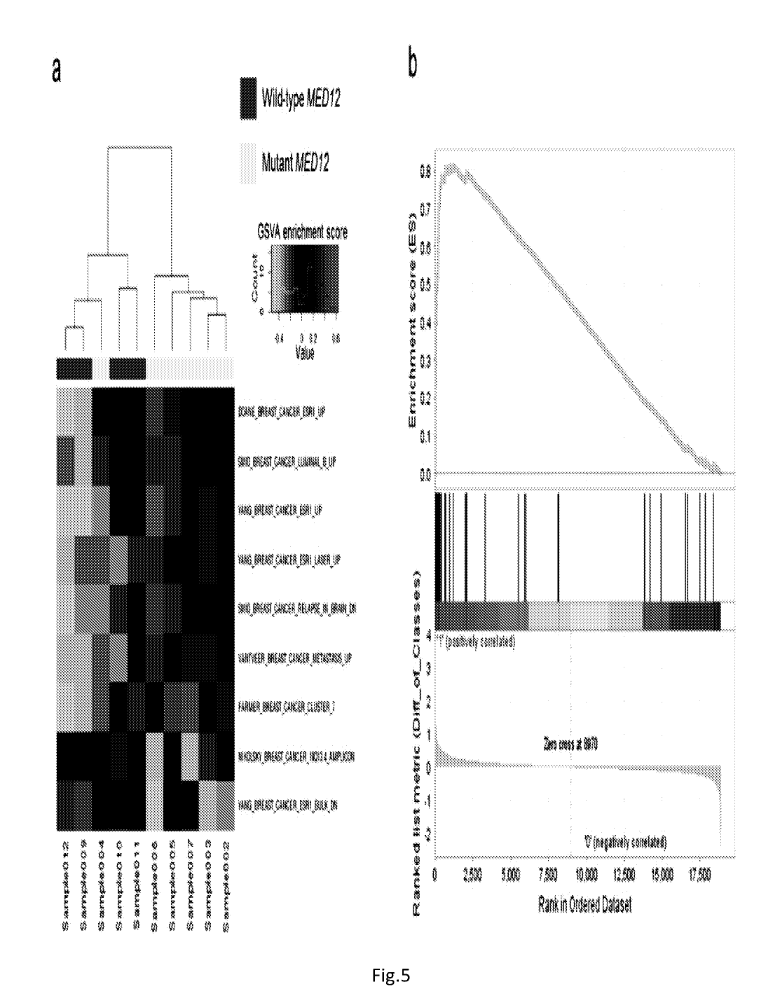

Results show that MED12 mutations are only found in the stromal compartment, and that epithelial portions of the FA tumor contained only wild-type as in FIG. 4. Frequent MED12 exon 2 somatic mutations have hitherto been found only in uterine leiomyoma (UL)¹³. The point mutations found in FA are remarkably similar to that of UL both in location and variant codon preference as indicated in Table 5. Both tumors are dominated by frequent codon 44 missense mutations (42% in FA and 49% in UL, p=0.28, two-tailed Fisher's exact test). Codon 36 missense mutations were the second-most frequent in both tumors and occurred at similar frequencies (4.1% in FA and 5% in UL, p=1.00, two-tailed Fisher's exact test). The present disclosure also observed codon 43 mutations and intronic T>A aberrant splice acceptor site mutation previously observed in UL. Altogether, every single point mutation in MED12 exon 2 detected in UL was also detected in FA. Additionally, both tumors also share a preference for in-frame deletions. These observations suggest that FAs and ULs may have a common underlying genetic basis.

### Example 7

Total RNA was extracted from 10 fresh frozen fibroadenoma tumors using Trizol (Invitrogen) and purified using the RNeasy mini kit (Qiagen). 10 μg of purified total RNA was then labelled according to standard Affymetrix protocol and then hybridized to Affymetrix GeneChip Human Genome U133 Plus 2.0 microarrays. Scanning of the microarrays was performed using the Affymetrix GeneChip Scanner 7G. CEL files were loaded into the R statistical environment (version 2.15.2) using the simpleaffy package³¹ and preprocessed using the robust multi-array average (RMA) algorithm³² with quantile normalization. Mapping of Affymetrix probe sets to genes was performed using the BrainArray custom CDF³³ (chip definition file) version 17. Differentially expressed genes between mutant MED12 and wild-type MED12 samples were identified based on empirical Bayes moderated t-statistics calculated using the limina package³⁴. A list of genes differentially expressed over 1.5 fold in either direction and with a p-value less than 0.05 is presented in Table 7. P-values were not significant after adjusting for multiple hypotheses due to the limited sample size. The microarray data has been deposited in the Gene Expression Omnibus³⁵ (GEO accession ID: GSE55594).

TABLE 7
Differentially expressed genes between mutant and wild-type
MED12 fibroadenoma samples.

| Gene Symbol | log2 fold-change | t-statistic | p-value |
|---|---|---|---|
| MMP13 | 3.823 | 3.113 | 0.010 |
| TAT | 2.764 | 4.569 | 0.001 |
| RFX6 | 2.034 | 3.469 | 0.006 |
| ERP27 | 1.936 | 3.693 | 0.004 |
| CYP4X1 | 1.880 | 2.341 | 0.040 |
| SUSD5 | 1.874 | 2.560 | 0.027 |
| IL13RA2 | 1.826 | 2.838 | 0.017 |
| C12orf69 | 1.675 | 2.371 | 0.038 |
| KCNK15 | 1.607 | 3.345 | 0.007 |
| CPA3 | 1.388 | 2.624 | 0.024 |
| SOWAHA | 1.359 | 2.587 | 0.026 |
| ENTPD1 | 1.347 | 2.323 | 0.041 |
| ADRA2A | 1.334 | 3.579 | 0.005 |
| C1orf64 | 1.266 | 3.642 | 0.004 |
| FSIP1 | 1.246 | 2.278 | 0.045 |
| REEP1 | 1.242 | 2.561 | 0.027 |
| RHOH | 1.238 | 2.397 | 0.036 |
| TTC39A | 1.232 | 3.907 | 0.003 |
| RERGL | 1.015 | 3.002 | 0.013 |
| TUBB2B | 1.008 | 2.736 | 0.020 |
| ITGA8 | 1.007 | 2.634 | 0.024 |
| FAM70A | 1.004 | 2.421 | 0.035 |
| SLC19A2 | 0.967 | 3.469 | 0.006 |
| LTBP2 | 0.957 | 2.344 | 0.040 |
| HEPH | 0.925 | 2.549 | 0.028 |
| SYTL4 | 0.910 | 3.022 | 0.012 |
| NRIP3 | 0.908 | 2.398 | 0.036 |
| ZNF552 | 0.895 | 2.736 | 0.020 |
| PREX1 | 0.883 | 3.389 | 0.006 |
| TTC36 | 0.865 | 2.739 | 0.020 |
| MLPH | 0.863 | 3.054 | 0.012 |
| AZGP1 | 0.861 | 2.634 | 0.024 |
| LOC100507165 | 0.854 | 2.882 | 0.016 |
| TUBB2A | 0.827 | 2.635 | 0.024 |
| GEM | 0.826 | 3.464 | 0.006 |
| ECM2 | 0.824 | 3.441 | 0.006 |
| TSPAN2 | 0.811 | 2.321 | 0.042 |
| HOMER1 | 0.810 | 2.809 | 0.018 |
| C11orf96 | 0.805 | 2.951 | 0.014 |
| CASC1 | 0.791 | 5.394 | 0.000 |
| FOXA1 | 0.776 | 2.481 | 0.031 |
| CSRP2 | 0.762 | 2.289 | 0.044 |
| KIAA1467 | 0.751 | 3.039 | 0.012 |
| TSPAN7 | 0.748 | 3.837 | 0.003 |
| LOC729970 | 0.739 | 5.084 | 0.000 |
| ACP5 | 0.719 | 2.632 | 0.024 |
| RNF175 | 0.710 | 2.606 | 0.025 |
| LYPD6 | 0.708 | 2.926 | 0.014 |
| FGFR1OP | 0.702 | 2.763 | 0.019 |
| MUC1 | 0.678 | 2.906 | 0.015 |
| C10orf116 | 0.675 | 2.704 | 0.021 |
| GPR160 | 0.663 | 2.387 | 0.037 |
| FRK | 0.657 | 2.273 | 0.045 |
| FJX1 | 0.656 | 2.255 | 0.047 |
| ECI2 | 0.645 | 3.662 | 0.004 |
| STK17A | 0.642 | 3.037 | 0.012 |
| SNX10 | 0.636 | 2.965 | 0.013 |
| TPBG | 0.631 | 2.939 | 0.014 |
| CDC42EP3 | 0.624 | 3.780 | 0.003 |
| C7orf10 | 0.613 | 2.583 | 0.026 |
| RAB38 | 0.608 | 2.268 | 0.046 |
| MYRIP | 0.608 | 2.675 | 0.022 |
| WWP1 | 0.603 | 2.413 | 0.035 |
| HS3ST1 | 0.594 | 2.337 | 0.040 |
| SLC25A16 | 0.592 | 2.741 | 0.020 |
| LOC100506100 | 0.586 | 3.252 | 0.008 |
| MFSD4 | −0.586 | −2.410 | 0.036 |
| PIK3R1 | −0.586 | −2.563 | 0.027 |
| PPAP2B | −0.598 | −2.482 | 0.031 |
| EMILIN3 | −0.601 | −2.455 | 0.033 |
| HLF | −0.605 | −3.216 | 0.009 |
| ARHGAP26 | −0.612 | −2.611 | 0.025 |
| IGSF5 | −0.613 | −2.310 | 0.042 |
| HOXA13 | −0.620 | −2.603 | 0.025 |
| PRKX | −0.626 | −2.635 | 0.024 |
| PPP3CA | −0.627 | −2.645 | 0.024 |
| DDR2 | −0.638 | −2.400 | 0.036 |
| FCRLB | −0.640 | −3.497 | 0.005 |
| UNC80 | −0.655 | −2.471 | 0.032 |
| RHOV | −0.657 | −2.554 | 0.028 |
| ZFHX4 | −0.664 | −2.437 | 0.034 |
| CD44 | −0.700 | −2.860 | 0.016 |
| FUT9 | −0.700 | −2.256 | 0.046 |
| TPTE2P6 | −0.703 | −2.450 | 0.033 |
| CXCR4 | −0.705 | −2.345 | 0.040 |
| EYA4 | −0.723 | −2.551 | 0.028 |
| HSD17B1 | −0.730 | −2.409 | 0.036 |
| FAM19A5 | −0.745 | −2.928 | 0.014 |
| C1orf51 | −0.757 | −2.361 | 0.039 |
| ITIH5 | −0.761 | −2.684 | 0.022 |
| FZD7 | −0.775 | −3.074 | 0.011 |
| CPE | −0.796 | −2.772 | 0.019 |
| OOEP | −0.844 | −3.102 | 0.011 |
| FAM13A | −0.860 | −3.198 | 0.009 |
| MNX1 | −0.863 | −4.462 | 0.001 |
| EMR2 | −0.867 | −4.237 | 0.002 |
| ITGA6 | −0.878 | −2.298 | 0.043 |
| MFAP3L | −0.881 | −2.450 | 0.033 |
| MAGEL2 | −0.883 | −2.400 | 0.036 |
| EBF3 | −0.890 | −2.720 | 0.021 |
| ADAMTSL3 | −0.933 | −2.340 | 0.040 |
| LOC158434 | −0.980 | −2.483 | 0.031 |
| ST8SIA1 | −1.003 | −2.898 | 0.015 |
| PDE9A | −1.051 | −2.601 | 0.026 |
| UG0898H09 | −1.051 | −2.624 | 0.024 |
| SLIT2 | −1.179 | −2.460 | 0.033 |
| SLC12A2 | −1.222 | −2.555 | 0.028 |
| CXCL1 | −1.278 | −2.222 | 0.049 |
| RYR3 | −1.358 | −2.419 | 0.035 |
| FGF10 | −1.380 | −2.932 | 0.014 |
| DDX43 | −1.384 | −4.831 | 0.001 |
| MFAP5 | −1.391 | −2.294 | 0.044 |
| NRK | −1.731 | −4.317 | 0.001 |
| SOX8 | −1.741 | −2.500 | 0.030 |
| PTH2R | −1.762 | −2.346 | 0.040 |
| GPC3 | −1.958 | −3.516 | 0.005 |
| ZFPM2 | −1.962 | −2.284 | 0.044 |
| LOC100652994 | −1.994 | −2.425 | 0.035 |
| LTF | −1.996 | −2.588 | 0.026 |

### Example 8

To characterize transcriptional changes associated with aberrant MED12, the present disclosure generated and compared the gene expression profiles of six MED12 mutated fibroadenoma samples against four MED12 wild-type fibroadenomas. Due to the limited sample size and fibroepithelial nature of fibroadenomas, the present disclosure used GSEA¹⁹ (Gene Set Enrichment Analysis) in order to identify potentially dysregulated pathways. Genes were rank-ordered by fold-change between MED12-mutant and wild-type fibroadenomas and subjected to GSEA against MSigDB¹⁹ (Molecular Signatures Database) curated (c2) gene sets. Particularly, the present disclosure integrated our gene expression data with publicly-available gene expression data of UL tumors (GEO accession ID: GSE30673). Lists of genes upregulated two-fold and four-fold were obtained by calculating fold-change of averages between mutant MED12 (n=8) and wild-type UL samples (n=2). To calculate if the overlap between genes upregulated in MED12-mutant fibroadenoma and MED12-mutant UL is significant, the present disclosure used the Gene Set Enrichment Analysis (GSEA) tool¹⁹. Briefly, genes in the fibroadenoma dataset were ranked-ordered according to log fold-change. The GSEA algorithm then examines where genes upregulated in UL fall in the rank-ordered list, and generates an enrichment score corresponding to how enriched a gene set is in either extreme end of the rank-ordered list as can be seen in FIG. 5b. Random, size-matched gene sets are then used to generate an empirical p-value. Similarly, GSEA analysis was also performed on our fibroadenoma microarray dataset against the MSigDB c2 (curated) gene sets¹⁹, which are derived from publications, canonical pathways and expert knowledge.

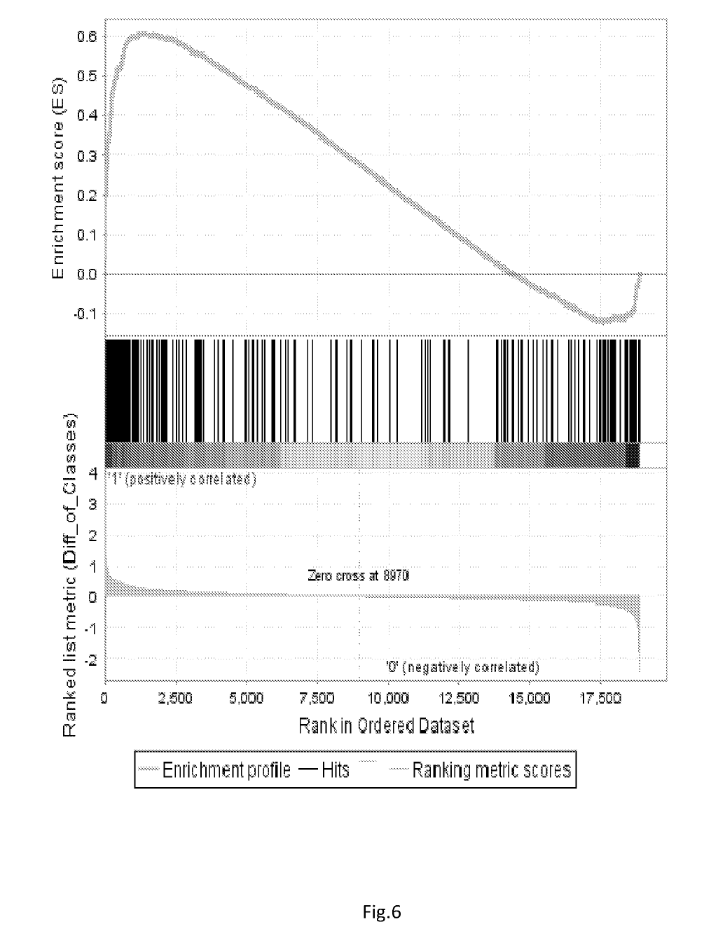

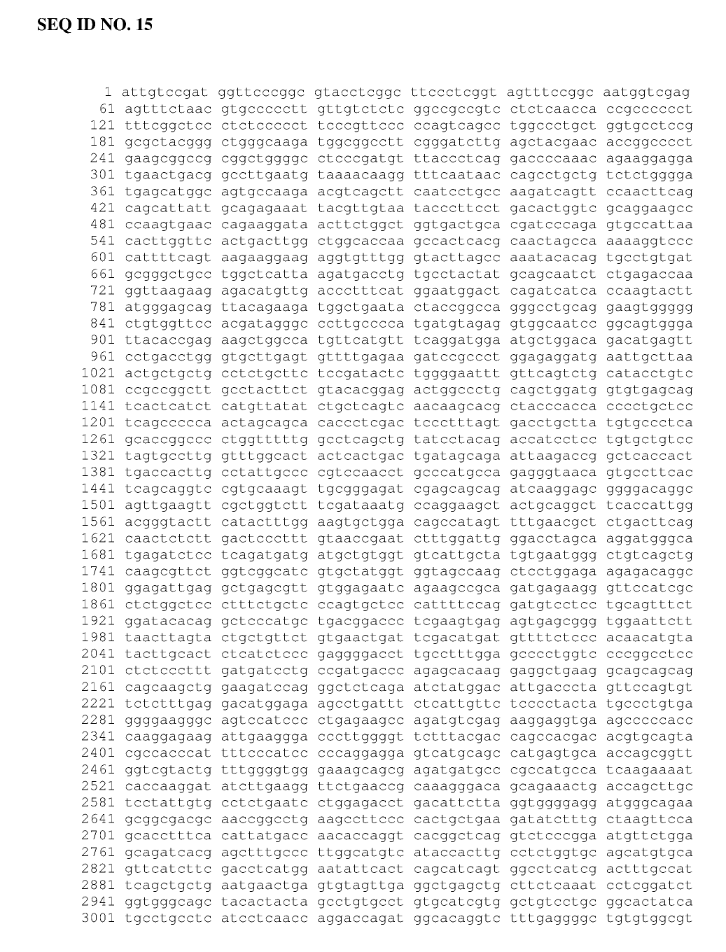

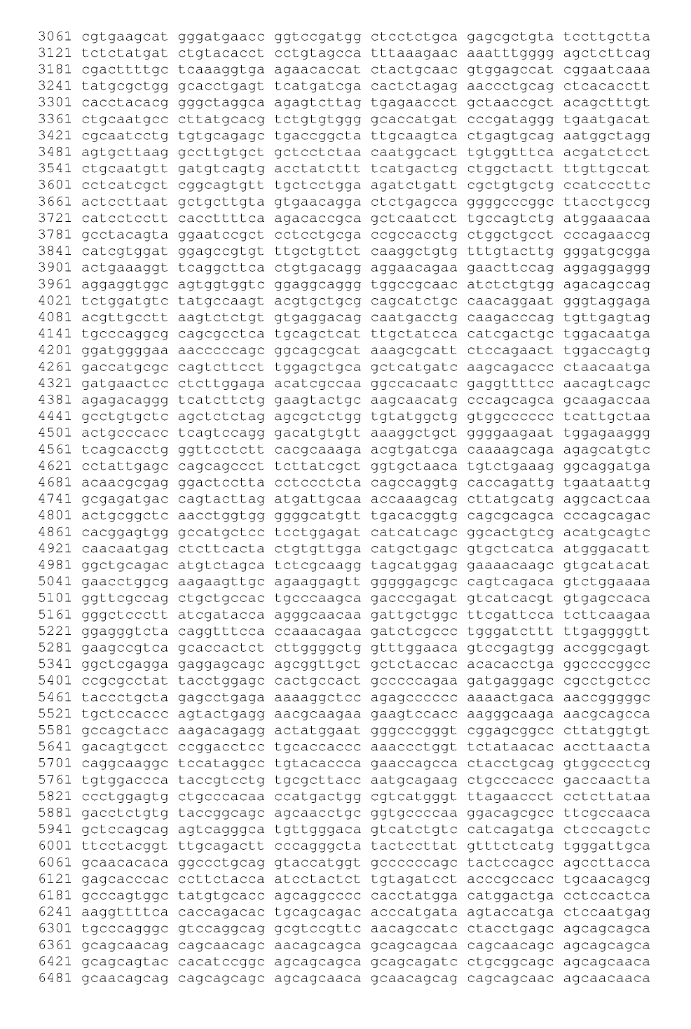

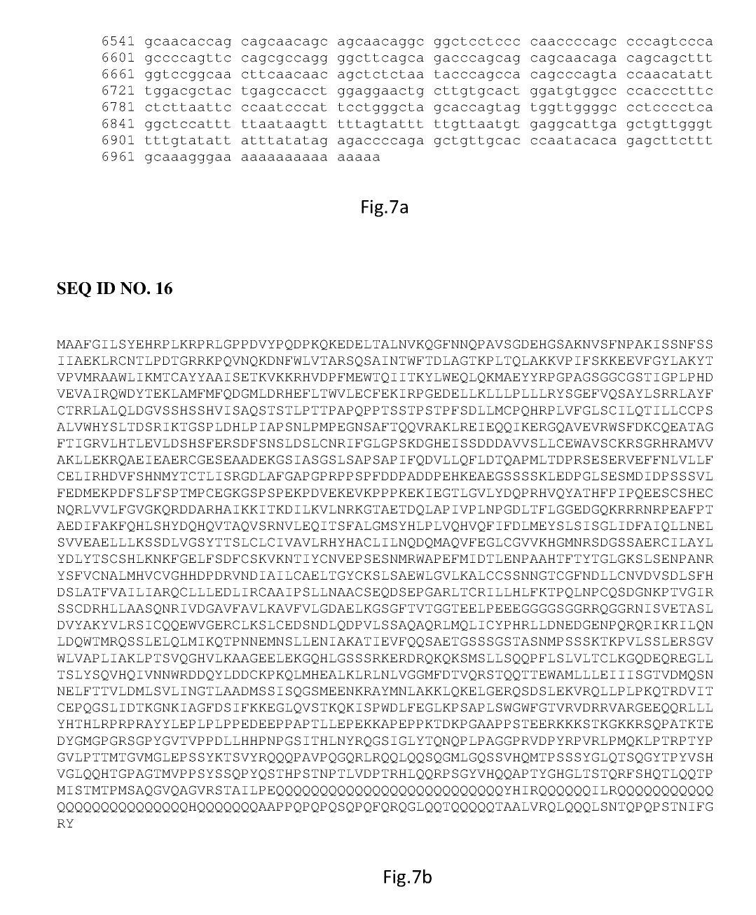

A list of candidate mutant MED12 target genes was obtained from the core-enriched genes (FIG. 6) in the GSEA analysis of our FA microarray data against upregulated genes in the UL dataset. Core-enriched genes are defined as those in the leading edge subset of the gene set (i.e. those that contributed most to the enrichment score). These genes were then used as input in the MSigDB web site ‘Compute Overlaps’ tool (accessed on 10 Feb. 2014, see URLs). Two classes of gene sets were used in the analysis; c2 (curated) and c5 (gene ontology). Given a gene list, the tool uses the hypergeometric test to compare it against gene sets to determine if the overlap exceeds chance. Gene sets with FDR³³ (false discovery rate) q-values<0.05 were considered to be significantly over-represented with members of the input gene list.

In order to study relative pathway activity on the level of individual samples, the present disclosure used the Gene Set Variation Analysis (GSVA) method³⁶. Using a non-parametric approach, GSVA transforms a gene by sample matrix into a gene set by sample matrix, facilitating the identification of differential activation of functionally related genes. Empirical Bayes moderated t-statistics³⁴ were then calculated and gene sets with p-values<0.05 were considered to have significantly differential activity between mutant MED12 and wild-type MED12 samples. GSVA was performed on two groups of gene sets. MSigDB c2 gene sets associated with breast cancer and estrogen signalling were considered, as shown in FIG. 5b. Unsupervised clustering of samples and gene sets in heatmaps was performed using the gplots package in R using a Euclidean distance metric and complete-linkage clustering.

Among others, genes upregulated in MED12-mutant fibroadenomas are associated with ER+ breast cancers, estrogen stimulus in ER+ breast cancer cells, extracellular matrix (ECM) regulation and TGFβ signalling as revealed in Table 8 and 9. As the top GSEA results suggested an association between MED12 mutations and activated estrogen signalling, the present disclosure performed GSVA (Gene Set Variation Analysis) on our microarrays to detect differential pathway activity between samples.

TABLE 8
Top 50 enriched MSigDB curated (c2) gene sets for genes upregulated in MED12 mutant
FA. Gene sets of interest are highlighted. ES: Enrichment Score, NES: Normalized Enrichment
Score, FDR: False Discovery Rate

| NAME | SIZE | ES | NES | FDR |
|---|---|---|---|---|
| DOANE_BREAST_CANCER_ESR1_UP | 103 | 0.801 | 2.826 | 0.000 |
| SMID_BREAST_CANCER_RELAPSE_IN_BRAIN_DN | 70 | 0.754 | 2.531 | 0.000 |
| SMID_BREAST_CANCER_RELAPSE_IN_BONE_UP | 86 | 0.724 | 2.526 | 0.000 |
| SMID_BREAST_CANCER_BASAL_DN | 597 | 0.579 | 2.508 | 0.000 |
| LIEN_BREAST_CARCINOMA_METAPLASTIC_VS_DUCTAL_DN | 94 | 0.705 | 2.472 | 0.000 |
| SMID_BREAST_CANCER_LUMINAL_B_UP | 153 | 0.638 | 2.419 | 0.000 |
| YANG_BREAST_CANCER_ESR1_UP | 33 | 0.821 | 2.413 | 0.000 |
| VANTVEER_BREAST_CANCER_ESR1_UP | 132 | 0.642 | 2.373 | 0.000 |
| NAGASHIMA_EGF_SIGNALING_UP | 50 | 0.738 | 2.310 | 0.000 |
| MASSARWEH_RESPONSE_TO_ESTRADIOL | 51 | 0.697 | 2.196 | 0.000 |
| NAGASHIMA_NRG1_SIGNALING_UP | 157 | 0.579 | 2.185 | 0.000 |
| REACTOME_DEGRADATION_OF_THE_EXTRACELLULAR_MATRIX | 26 | 0.765 | 2.140 | 0.000 |
| CHIBA_RESPONSE_TO_TSA_UP | 49 | 0.685 | 2.135 | 0.000 |
| POOLA_INVASIVE_BREAST_CANCER_DN | 123 | 0.580 | 2.124 | 0.001 |
| CHARAFE_BREAST_CANCER_LUMINAL_VS_BASAL_UP | 314 | 0.509 | 2.088 | 0.003 |
| DORN_ADENOVIRUS_INFECTION_48HR_DN | 34 | 0.698 | 2.075 | 0.003 |
| COWLING_MYCN_TARGETS | 36 | 0.692 | 2.074 | 0.003 |
| PID_UPA_UPAR_PATHWAY | 38 | 0.699 | 2.072 | 0.003 |
| AMIT_SERUM_RESPONSE_60_MCF10A | 53 | 0.643 | 2.061 | 0.003 |
| VANTVEER_BREAST_CANCER_METASTASIS_UP | 43 | 0.660 | 2.039 | 0.005 |
| WANG_TNF_TARGETS | 23 | 0.747 | 2.017 | 0.007 |
| AMIT_EGF_RESPONSE_40_HELA | 38 | 0.672 | 2.003 | 0.009 |
| PLASARI_TGFB1_TARGETS_1HR_UP | 30 | 0.693 | 2.002 | 0.009 |
| LIM_MAMMARY_LUMINAL_MATURE_UP | 106 | 0.563 | 2.000 | 0.009 |
| DORN_ADENOVIRUS_INFECTION_32HR_DN | 33 | 0.670 | 1.992 | 0.010 |
| FARMER_BREAST_CANCER_BASAL_VS_LULMINAL | 291 | 0.487 | 1.985 | 0.011 |
| SMID_BREAST_CANCER_LUMINAL_A_UP | 81 | 0.578 | 1.981 | 0.011 |
| MASSARWEH_TAMOXIFEN_RESISTANCE_DN | 201 | 0.507 | 1.977 | 0.011 |
| NIELSEN_LEIOMYOSARCOMA_CNN1_UP | 18 | 0.765 | 1.971 | 0.013 |
| AMIT_EGF_RESPONSE_40_MCF10A | 18 | 0.768 | 1.954 | 0.016 |
| FRASOR_RESPONSE_TO_ESTRADIOL_UP | 35 | 0.654 | 1.952 | 0.016 |
| REACTOME_EXTRACELLULAR_MATRIX_ORGANIZATION | 83 | 0.554 | 1.950 | 0.016 |
| YANG_BREAST_CANCER_ESR1_BULK_UP | 19 | 0.741 | 1.948 | 0.016 |
| WATTEL_AUTONOMOUS_THYROID_ADENOMA_DN | 50 | 0.611 | 1.948 | 0.016 |
| PHONG_TNF_TARGETS_UP | 60 | 0.589 | 1.948 | 0.015 |
| SU_THYMUS | 19 | 0.755 | 1.946 | 0.015 |
| DUTERTRE_ESTRADIOL_RESPONSE_24HR_UP | 290 | 0.474 | 1.941 | 0.016 |
| WILSON_PROTEASES_AT_TUMOR_BONE_INTERFACE_UP | 21 | 0.731 | 1.933 | 0.018 |
| ROSTY_CERVICAL_CANCER_PROLIFERATION_CLUSTER | 127 | 0.521 | 1.932 | 0.017 |
| PLASARI_TGFB1_TARGETS_10HR_UP | 182 | 0.503 | 1.932 | 0.017 |
| MCMURRAY_TP53_HRAS_COOPERATION_RESPONSE_DN | 61 | 0.586 | 1.920 | 0.020 |
| UZONYI_RESPONSE_TO_LEUKOTRIENE_AND_THROMBIN | 34 | 0.654 | 1.915 | 0.021 |
| DIRMEIER_LMP1_RESPONSE_EARLY | 61 | 0.591 | 1.911 | 0.022 |
| DAZARD_UV_RESPONSE_CLUSTER_G4 | 17 | 0.746 | 1.909 | 0.022 |
| JAZAERI_BREAST_CANCER_BRCA1_VS_BRCA2_DN | 37 | 0.641 | 1.904 | 0.023 |
| TIAN_TNF_SIGNALING_NOT_VIA_NFKB | 21 | 0.716 | 1.897 | 0.025 |
| CREIGHTON_ENDOCRINE_THERAPY_RESISTANCE_4 | 245 | 0.468 | 1.893 | 0.026 |
| WANG_RESPONSE_TO_FORSKOLIN_UP | 21 | 0.696 | 1.872 | 0.033 |
| TRAYNOR_RETT_SYNDROM_UP | 41 | 0.608 | 1.869 | 0.034 |
| KORKOLA_TERATOMA | 34 | 0.635 | 1.869 | 0.033 |

TABLE 9
Top 50 enriched MSigDB curated (c2) gene sets for genes downregulated in
MED12 mutant FA. Gene sets of interest are highlighted. ES: Enrichment Score, NES:
Normalized Enrichment Score, FDR: False Discovery Rate

| NAME | SIZE | ES | NES | FDR |
|---|---|---|---|---|
| LIM_MAMMARY_LUMINAL_PROGENITOR_UP | 53 | −0.718 | −2.468 | 0.000 |
| SMID_BREAST_CANCER_RELAPSE_IN_BONE_DN | 283 | −0.517 | −2.293 | 0.001 |
| DOANE_BREAST_CANCER_ESR1_DN | 46 | −0.685 | −2.239 | 0.001 |
| SMID_BREAST_CANCER_LUMINAL_B_DN | 500 | −0.459 | −2.113 | 0.010 |
| SMID_BREAST_CANCER_BASAL_UP | 581 | −0.452 | −2.093 | 0.014 |
| ONDER_CDH1_TARGETS_3_DN | 50 | −0.611 | −2.052 | 0.023 |
| YANG_BREAST_CANCER_ESR1_DN | 24 | −0.696 | −1.988 | 0.048 |
| REACTOME_LATENT_INFECTION_OF_HOMO_SAPIENS_WITH_MYCOBACTERIUM_TUBERCULOSIS | 30 | −0.654 | −1.948 | 0.074 |
| CHIBA_RESPONSE_TO_TSA_DN | 21 | −0.699 | −1.917 | 0.101 |
| KEGG_GLYCOSPHINGOLIPID_BIOSYNTHESIS_LACTO_AND_NEOLACTO_SERIES | 26 | −0.649 | −1.891 | 0.126 |
| KEGG_LONG_TERM_POTENTIATION | 64 | −0.532 | −1.880 | 0.132 |
| YANG_BREAST_CANCER_ESR1_BULK_DN | 19 | −0.679 | −1.867 | 0.142 |
| CHIARADONNA_NEOPLASTIC_TRANSFORMATION_CDC25_UP | 110 | −0.481 | −1.842 | 0.177 |
| BIOCARTA_CXCR4_PATHWAY | 22 | −0.654 | −1.838 | 0.172 |
| PID_A6B1_A6B4_INTEGRIN_PATHWAY | 44 | −0.565 | −1.837 | 0.162 |
| BIOCARTA_IL7_PATHWAY | 17 | −0.699 | −1.816 | 0.194 |
| LEE_LIVER_CANCER_DENA_UP | 58 | −0.517 | −1.815 | 0.184 |
| ROY_WOUND_BLOOD_VESSEL_DN | 20 | −0.649 | −1.815 | 0.175 |
| LIM_MAMMARY_LUMINAL_MATURE_DN | 89 | −0.481 | −1.799 | 0.199 |
| REACTOME_INTERACTION_BETWEEN_L1_AND_ANKYRINS | 20 | −0.654 | −1.790 | 0.207 |
| REACTOME_ACTIVATED_POINT_MUTANTS_OF_FGFR2 | 16 | −0.666 | −1.787 | 0.203 |
| KEGG_AXON_GUIDANCE | 124 | −0.453 | −1.786 | 0.196 |
| NAKAYAMA_SOFT_TISSUE_TUMORS_PCA2_DN | 75 | −0.486 | −1.773 | 0.215 |
| KEGG_ALZHEIMERS_DISEASE | 142 | −0.440 | −1.762 | 0.230 |
| REACTOME_TRAFFICKING_OF_AMPA_RECEPTORS | 26 | −0.601 | −1.753 | 0.241 |
| PID_NCADHERINPATHWAY | 30 | −0.585 | −1.751 | 0.238 |
| CHEN_LVAD_SUPPORT_OF_FAILING_HEART_UP | 93 | −0.465 | −1.750 | 0.231 |
| KEGG_SMALL_CELL_LUNG_CANCER | 79 | −0.481 | −1.746 | 0.231 |
| NIELSEN_SCHWANNOMA_UP | 15 | −0.683 | −1.744 | 0.228 |
| SUZUKI_RESPONSE_TO_TSA_AND_DECITABINE_1A | 19 | −0.629 | −1.740 | 0.228 |
| CHEMELLO_SOLEUS_VS_EDL_MYOFIBERS_DN | 19 | −0.637 | −1.737 | 0.229 |
| JOHNSTONE_PARVB_TARGETS_1_DN | 43 | −0.533 | −1.731 | 0.235 |
| PID_INTEGRIN1_PATHWAY | 63 | −0.489 | −1.731 | 0.228 |
| TIEN_INTESTINE_PROBIOTICS_6HR_UP | 39 | −0.542 | −1.726 | 0.232 |
| REACTOME_SIGNALING_BY_INSULIN_RECEPTOR | 98 | −0.446 | −1.724 | 0.229 |
| REACTOME_NEPHRIN_INTERACTIONS | 19 | −0.641 | −1.723 | 0.225 |
| GUILLAUMOND_KLF10_TARGETS_DN | 24 | −0.608 | −1.718 | 0.231 |
| JAEGER_METASTASIS_DN | 234 | −0.399 | −1.716 | 0.230 |
| REACTOME_UNBLOCKING_OF_NMDA_RECEPTOR_GLUTAMATE_BINDING_AND_ACTIVATION | 15 | −0.685 | −1.708 | 0.242 |
| MAHADEVAN_RESPONSE_TO_MP470_UP | 19 | −0.606 | −1.699 | 0.255 |
| REACTOME_FGFR_LIGAND_BINDING_AND_ACTIVATION | 22 | −0.610 | −1.694 | 0.260 |
| LEE_LIVER_CANCER_E2F1_UP | 57 | −0.489 | −1.693 | 0.256 |
| CHIARADONNA_NEOPLASTIC_TRANSFORMATION_KRAS_UP | 112 | −0.444 | −1.693 | 0.251 |
| ONDER_CDH1_TARGETS_1_DN | 146 | −0.423 | −1.691 | 0.248 |
| VANTVEER_BREAST_CANCER_ESR1_DN | 206 | −0.408 | −1.691 | 0.243 |
| REACTOME_PI3K_CASCADE | 62 | −0.487 | −1.686 | 0.249 |
| PID_IL2_STAT5PATHWAY | 30 | −0.567 | −1.685 | 0.247 |
| KEGG_AMYOTROPHIC_LATERAL_SCLEROSIS_ALS | 47 | −0.503 | −1.683 | 0.245 |
| YANG_MUC2_TARGETS_DUODENUM_6MO_DN | 19 | −0.609 | −1.677 | 0.255 |
| KEGG_GLIOMA | 63 | −0.477 | −1.676 | 0.252 |

Given the similarity of the MED12 mutation spectrum in FAs and ULs, the present disclosure hypothesized the integration of FA and UL molecular data might allow further pinpointing of genes and pathways. Indeed, GSEA on our FA dataset against a previously-published set of genes upregulated in MED12-mutated ULs revealed a strong similarity of upregulated genes in MED12-mutated FAs and ULs. Specifically, genes upregulated in MED12-mutant FA samples were significantly enriched for genes upregulated over two-fold in MED12-mutant UL (enrichment score=0.61, p=0) as shown in FIG. 2, with enrichment becoming even more profound when only genes upregulated four-fold were considered (enrichment score=0.81, p=0), with reference to FIG. 5b. Analysis of core-enriched genes (i.e. genes commonly upregulated in both FA and UL with mutant MED12) revealed, as in Table 10 below, that they were over-represented with genes associated with extracellular matrix (ECM) organization, estrogen signalling, as well as TGFβ and Wnt signalling.

TABLE 10
MSigDB curated (c2) gene sets significantly overlapping with candidate

| Gene Set Name | Gene Set | # Genes in | FDR q- |
|---|---|---|---|
| RIGGI_EWING_SARCOMA_PROGENITOR_UP | 430 | 17 | 0.00E+00 |
| VECCHI_GASTRIC_CANCER_ADVANCED_VS_EARLY | 175 | 9 | 2.20E−08 |
| SCHUETZ_BREAST_CANCER_DUCTAL_INVASIVE_U | 351 | 11 | 2.20E−08 |
| TURASHVILI_BREAST_LOBULAR_CARCINOMA_VS_{—} | 74 | 7 | 4.50E−08 |
| WONG_ADULT_TISSUE_STEM_MODULE | 721 | 13 | 1.54E−07 |
| BENPORATH_SUZ12_TARGETS | 1038 | 14 | 1.01E−06 |
| BENPORATH_EED_TARGETS | 1062 | 14 | 1.15E−06 |
| CHANDRAN_METASTASIS_DN | 306 | 9 | 1.15E−06 |
| BENPORATH_ES_WITH_H3K27ME3 | 1118 | 14 | 1.74E−06 |
| YANG_BCL3_TARGETS_UP | 364 | 9 | 4.16E−06 |
| MARTORIATI_MDM4_TARGETS_NEUROEPITHELIUM | 164 | 7 | 4.50E−06 |
| CHIANG_LIVER_CANCER_SUBCLASS_CTNNB1_DN | 170 | 7 | 5.29E−06 |
| MCBRYAN_PUBERTAL_BREAST_4_5WK_UP | 271 | 8 | 5.74E−06 |
| SMID_BREAST_CANCER_BASAL_DN | 701 | 11 | 6.56E−06 |
| POOLA_INVASIVE_BREAST_CANCER_UP | 288 | 8 | 7.97E−06 |
| BOQUEST_STEM_CELL_CULTURED_VS_FRESH_UP | 425 | 9 | 9.83E−06 |
| PLASARI_TGFB1_TARGETS_10HR_UP | 199 | 7 | 1.10E−05 |
| GOZGIT_ESR1_TARGETS_DN | 781 | 11 | 1.52E−05 |
| CROMER_TUMORIGENESIS_UP | 63 | 5 | 1.64E−05 |
| TURASHVILI_BREAST_LOBULAR_CARCINOMA_VS_{—} | 69 | 5 | 2.39E−05 |
| ZWANG_TRANSIENTLY_UP_BY_2ND_EGF_PULSE_O | 1725 | 15 | 2.39E−05 |
| AFFAR_YY1_TARGETS_DN | 234 | 7 | 2.57E−05 |
| SABATES_COLORECTAL_ADENOMA_UP | 141 | 6 | 2.60E−05 |
| SENESE_HDAC3_TARGETS_UP | 501 | 9 | 2.65E−05 |
| REACTOME_DEGRADATION_OF_THE_EXTRACELLU | 29 | 4 | 2.81E−05 |
| LEE_NEURAL_CREST_STEM_CELL_UP | 146 | 6 | 2.81E−05 |
| MEISSNER_BRAIN_HCP_WITH_H3K4ME3_AND_H3K2 | 1069 | 12 | 2.81E−05 |
| MCLACHLAN_DENTAL_CARIES_UP | 253 | 7 | 3.43E−05 |
| MIYAGAWA_TARGETS_OF_EWSR1_ETS_FUSIONS_U | 259 | 7 | 3.88E−05 |
| SANA_TNF_SIGNALING_UP | 83 | 5 | 4.18E−05 |
| HAN_SATB1_TARGETS_UP | 395 | 8 | 4.27E−05 |
| REACTOME_SIGNALING_BY_GPCR | 920 | 11 | 4.27E−05 |
| SERVITJA_ISLET_HNF1A_TARGETS_UP | 163 | 6 | 4.27E−05 |
| GHANDHI_BYSTANDER_IRRADIATION_UP | 86 | 5 | 4.41E−05 |
| BENPORATH_SOX2_TARGETS | 734 | 10 | 4.47E−05 |
| REACTOME_GPCR_LIGAND_BINDING | 408 | 8 | 4.75E−05 |
| CHICAS_RB1_TARGETS_CONFLUENT | 567 | 9 | 4.84E−05 |
| WANG_SMARCE1_TARGETS_UP | 280 | 7 | 5.01E−05 |
| NUYTTEN_NIPP1_TARGETS_UP | 769 | 11 | 6.12E−05 |
| SCHAEFFER_PROSTATE_DEVELOPMENT_48HR_DN | 428 | 8 | 6.12E−05 |
| BROWNE_HCMV_INFECTION_48HR_UP | 180 | 6 | 6.15E−05 |
| GAUSSMANN_MLL_AF4_FUSION_TARGETS_E_UP | 97 | 5 | 6.52E−05 |
| KANG_IMMORTALIZED_BY_TERT_DN | 102 | 5 | 8.17E−05 |
| COWLING_MYCN_TARGETS | 43 | 4 | 8.26E−05 |
| JAEGER_METASTASIS_UP | 44 | 4 | 8.87E−05 |
| NUYTTEN_EZH2_TARGETS_UP | 1037 | 11 | 9.87E−05 |
| REACTOME_GASTRIN_CREB_SIGNALLING_PATHWA | 205 | 6 | 1.15E−04 |
| BENPORATH_PRC2_TARGETS | 652 | 9 | 1.16E−04 |
| MCCLUNG_DELTA_FOSB_TARGETS_2WK | 48 | 4 | 1.16E−04 |
| RIZKI_TUMOR_INVASIVENESS_3D_UP | 210 | 6 | 1.24E−04 |
| RODWELL_AGING_KIDNEY_UP | 487 | 8 | 1.26E−04 |
| MCBRYAN_PUBERTAL_BREAST_3_4WK_UP | 214 | 6 | 1.33E−04 |
| RODWELL_AGING_KIDNEY_NO_BLOOD_UP | 222 | 6 | 1.61E−04 |
| YAMASHITA_METHYLATED_IN_PROSTATE_CANCE | 57 | 4 | 2.12E−04 |
| ZHOU_INFLAMMATORY_RESPONSE_FIMA_UP | 544 | 8 | 2.65E−04 |
| ANASTASSIOU_CANCER_MESENCHYMAL_TRANSITI | 64 | 5 | 3.25E−04 |
| DOUGLAS_BMI1_TARGETS_UP | 566 | 8 | 3.41E−04 |
| KUMAR_TARGETS_OF_MLL_AF9_FUSION | 405 | 7 | 3.78E−04 |
| CUI_TCF21_TARGETS_2_UP | 428 | 7 | 5.33E−04 |
| CORRE_MULTIPLE_MYELOMA_UP | 74 | 4 | 5.34E−04 |
| NAKAYAMA_SOFT_TISSUE_TUMORS_PCA1_DN | 74 | 4 | 5.34E−04 |
| WANG_MLL_TARGETS | 289 | 6 | 6.18E−04 |
| DELYS_THYROID_CANCER_UP | 443 | 7 | 6.18E−04 |
| BENPORATH_OCT4_TARGETS | 290 | 6 | 6.18E−04 |
| SABATES_COLORECTAL_ADENOMA_DN | 291 | 6 | 6.21E−04 |
| WONG_ENDMETRIUM_CANCER_DN | 82 | 4 | 7.43E−04 |
| SMID_BREAST_CANCER_BASAL_UP | 648 | 8 | 7.76E−04 |
| ONDER_CDH1_TARGETS_2_DN | 464 | 7 | 7.80E−04 |
| KEGG_ECM_RECEPTOR_INTERACTION | 84 | 4 | 7.82E−04 |
| KEGG_TGF_BETA_SIGNALING_PATHWAY | 86 | 4 | 8.47E−04 |
| REACTOME_EXTRACELLULAR_MATRIX_ORGANIZA | 87 | 4 | 8.54E−04 |
| SASSON_RESPONSE_TO_GONADOTROPHINS_DN | 87 | 4 | 8.54E−04 |
| SMID_BREAST_CANCER_RELAPSE_IN_BONE_DN | 315 | 6 | 8.54E−04 |
| REACTOME_G_ALPHA_Q_SIGNALLING_EVENTS | 184 | 5 | 8.54E−04 |
| BYSTRYKH_HEMATOPOIESIS_STEM_CELL_QTL_TR | 882 | 9 | 8.54E−04 |
| SASSON_RESPONSE_TO_FORSKOLIN_DN | 88 | 4 | 8.54E−04 |
| ABE_VEGFA_TARGETS_30MIN | 29 | 3 | 9.07E−04 |
| SCHAEFFER_PROSTATE_DEVELOPMENT_48HR_UP | 487 | 7 | 9.28E−04 |
| RIGGI_EWING_SARCOMA_PROGENITOR_DN | 191 | 5 | 9.59E−04 |
| STAEGE_EWING_FAMILY_TUMOR | 33 | 3 | 1.30E−03 |
| RICKMAN_HEAD_AND_NECK_CANCER_A | 100 | 4 | 1.33E−03 |
| PLASARI_TGFB1_TARGETS_1HR_UP | 34 | 3 | 1.39E−03 |
| PEREZ_TP63_TARGETS | 355 | 6 | 1.48E−03 |
| LI_CISPLATIN_RESISTANCE_DN | 35 | 3 | 1.48E−03 |
| WIERENGA_STAT5A_TARGETS_UP | 217 | 5 | 1.64E−03 |
| IZADPANAH_STEM_CELL_ADIPOSE_VS_BONE_DN | 108 | 4 | 1.67E−03 |
| WESTON_VEGFA_TARGETS | 108 | 4 | 1.67E−03 |
| CUI_TCF21_TARGETS_UP | 37 | 3 | 1.67E−03 |
| BLALOCK_ALZHEIMERS_DISEASE_DN | 1237 | 10 | 1.73E−03 |
| GHANDHI_DIRECT_IRRADIATION_UP | 110 | 4 | 1.74E−03 |
| LABBE_TARGETS_OF_TGFB1_AND_WNT3A_UP | 111 | 4 | 1.78E−03 |
| SMID_BREAST_CANCER_LUMINAL_B_DN | 564 | 7 | 2.00E−03 |
| SCHAEFFER_PROSTATE_DEVELOPMENT_12HR_UP | 116 | 4 | 2.07E−03 |
| CERVERA_SDHB_TARGETS_1_UP | 118 | 4 | 2.17E−03 |
| MARKEY_RB1_CHRONIC_LOF_DN | 118 | 4 | 2.17E−03 |
| PID_THROMBIN_PAR1_PATHWAY | 43 | 3 | 2.42E−03 |
| YOSHIMURA_MAPK8_TARGETS_UP | 1305 | 10 | 2.46E−03 |
| HOOI_ST7_TARGETS_DN | 123 | 4 | 2.46E−03 |
| PLASARI_TGFB1_TARGETS_10HR_DN | 244 | 5 | 2.46E−03 |
| MARTINEZ_RB1_AND_TP53_TARGETS_DN | 591 | 7 | 2.47E−03 |

Moreover, previous study reported that one out of eight (12.5%) fibroadenomas exhibited a non-silent TP53 mutation¹⁰, whereas another study reported no somatic TP53 mutations in fibroadenomas from women who remained unaffected by breast cancer after an average follow-up of ten years¹¹. A single PIK3CA mutation has also been reported from a screen of ten fibroadenoma tumors¹². It is likely that those cases that harbour PIK3CA and TP53 mutations may actually indicate more aggressive phylloides tumors³⁷ (subtype of fibroepithelial tumors) rather than the true benign fibroadenomas. Therefore the presence of a single genetic alteration, in the absence of others such as P53 or PIK3CA, may be a more accurate biomarker for benign fibroadenoma. Therefore, early identification of such genetic attributes or methods allowing one to discover the likelihood of genetic deficiencies is greatly desired.

Candidate aberrant MED12 target genes were also enriched for genes downregulated in liver cancer with activated beta-catenin (CTNNB1). As MED12 plays a vital role in transducing Wnt/beta-catenin signaling²⁰, this observation is consistent with MED12 mutations resulting in aberrant beta-catenin signalling, which is involved in regulating focal adhesion. Accordingly, GO (gene ontology) analysis showed that genes upregulated in mutant MED12 samples were over-represented with those expressed in the extracellular region as shown in Table 11.

TABLE 11
MSigDB GO (c5) gene sets significantly overlapping with candidate mutant
MED12 target genes.

| | Gene | # Genes in | |
|---|---|---|---|
| | Set | Overlap | FDR |
| Gene Set Name | Size (K) | (k) | q-value |
| EXTRACELLULAR_REGION_PART | 338 | 13 | 1.79E−11 |
| EXTRACELLULAR_REGION | 447 | 13 | 3.12E−10 |
| EXTRACELLULAR_SPACE | 245 | 9 | 1.35E−07 |
| MULTICELLULAR_ORGANISMAL_DEVELOPMENT | 1049 | 13 | 5.28E−06 |
| PROTEINACEOUS_EXTRACELLULAR_MATRIX | 98 | 5 | 1.59E−04 |
| EXTRACELLULAR_MATRIX | 100 | 5 | 1.59E−04 |
| ANATOMICAL_STRUCTURE_DEVELOPMENT | 1013 | 11 | 1.59E−04 |
| REGULATION_OF_BIOLOGICAL_QUALITY | 419 | 7 | 1.05E−03 |
| INTEGRAL_TO_MEMBRANE | 1330 | 11 | 1.44E−03 |
| SYSTEM_DEVELOPMENT | 861 | 9 | 1.44E−03 |
| INTRINSIC_TO_MEMBRANE | 1348 | 11 | 1.44E−03 |
| METALLOENDOPEPTIDASE_ACTIVITY | 27 | 3 | 1.44E−03 |
| CELL_FRACTION | 493 | 7 | 1.85E−03 |
| REGULATION_OF_SIGNAL_TRANSDUCTION | 222 | 5 | 3.42E−03 |
| ORGAN_DEVELOPMENT | 571 | 7 | 4.09E−03 |
| TRANSMEMBRANE_RECEPTOR_ACTIVITY | 418 | 6 | 5.89E−03 |
| METALLOPEPTIDASE_ACTIVITY | 50 | 3 | 6.26E−03 |
| MEMBRANE_PART | 1670 | 11 | 6.26E−03 |
| HYDROLASE_ACTIVITY_ACTING_ON_ESTER_BONDS | 269 | 5 | 6.26E−03 |
| MEMBRANE | 1994 | 12 | 6.46E−03 |
| SOLUBLE_FRACTION | 161 | 4 | 1.01E−02 |
| RESPONSE_TO_EXTERNAL_STIMULUS | 312 | 5 | 1.08E−02 |
| AXON | 12 | 2 | 1.08E−02 |
| INTEGRAL_TO_PLASMA_MEMBRANE | 977 | 8 | 1.18E−02 |
| INTRINSIC_TO_PLASMA_MEMBRANE | 991 | 8 | 1.25E−02 |
| PROTEOLYSIS | 191 | 4 | 1.56E−02 |
| HOMEOSTATIC_PROCESS | 207 | 4 | 2.02E−02 |
| RECEPTOR_ACTIVITY | 583 | 6 | 2.02E−02 |
| CATION_BINDING | 213 | 4 | 2.11E−02 |
| CELLULAR_PROTEIN_METABOLIC_PROCESS | 1117 | 8 | 2.25E−02 |
| MUSCLE_DEVELOPMENT | 93 | 3 | 2.25E−02 |
| CELLULAR_MACROMOLECULE_METABOLIC_PROCESS | 1131 | 8 | 2.25E−02 |
| PLASMA_MEMBRANE | 1426 | 9 | 2.25E−02 |
| CELL_MIGRATION | 96 | 3 | 2.25E−02 |
| NEURON_PROJECTION | 21 | 2 | 2.25E−02 |
| PLASMA_MEMBRANE_PART | 1158 | 8 | 2.43E−02 |
| CELL_SURFACE_RECEPTOR_LINKED_SIGNAL_TRANSDUCTION_GO_0007166 | 641 | 6 | 2.49E−02 |
| REGULATION_OF_G_PROTEIN_COUPLED_RECEPTOR_PROTEIN_SIGNALING_PATHWAY | 23 | 2 | 2.49E−02 |
| CELLULAR_CATION_HOMEOSTASIS | 106 | 3 | 2.66E−02 |
| CATION_HOMEOSTASIS | 109 | 3 | 2.81E−02 |
| PROTEIN_METABOLIC_PROCESS | 1231 | 8 | 3.17E−02 |
| ENDOPEPTIDASE_ACTIVITY | 117 | 3 | 3.29E−02 |
| ION_BINDING | 273 | 4 | 3.60E−02 |
| ION_HOMEOSTASIS | 129 | 3 | 4.16E−02 |
| SIGNAL_TRANSDUCTION | 1634 | 9 | 4.37E−02 |
| RHODOPSIN_LIKE_RECEPTOR_ACTIVITY | 134 | 3 | 4.44E−02 |
| ENZYME_LINKED_RECEPTOR_PROTEIN_SIGNALING_PATHWAY | 140 | 3 | 4.92E−02 |

It is crucial to note that the present disclosure is implementable to detect constitutional mutations regarding MED12 gene mutation, more particularly mutations located in exon 2 of MED12 gene, despite the subjects experimented in the foregoing examples may have obtained these mutations as somatic mutations. It is to be understood also that the present invention may be embodied in other specific forms and is not limited to the sole embodiment described above. However modification and equivalents of the disclosed concepts such as those which readily occur to one skilled in the art are intended to be included within the scope of the claims which are appended thereto

### REFERENCES

1. Krishnamurthy, S., Ashfaq, R., Shin, H. J. C. & Sneige, N. Distinction of phyllodes tumor from fibroadenoma. Cancer Cytopathol. 90, 342-349 (2000).
2. Fine, R. E. et al. Low-risk palpable breast masses removed using a vacuum-assisted handheld device. Am. J. Surg. 186, 362-367 (2003).
3. Bernardes, J. R. M., Jr, Seixas, M. T., Lima, G. R., Marinho, L. C. & Gebrim, L. H. The effect of tamoxifen on PCNA expression in fibroadenomas. Breast J. 9, 302-306 (2003).
4. Coriaty Nelson, Z., Ray, R. M., Gao, D. L. & Thomas, D. B. Risk factors for fibroadenoma in a cohort of female textile workers in Shanghai, China. Am. J. Epidemiol. 156, 599-605 (2002).
5. Noguchi, S., Motomura, K., Inaji, H., Imaoka, S. & Koyama, H. Clonal Analysis of Fibroadenoma and Phyllodes Tumor of the Breast. Cancer Res. 53, 4071-4074 (1993).
6. Dupont, W. D. et al. Long-term risk of breast cancer in women with fibroadenoma. N. Engl. J. Med. 331, 10-15 (1994).
7. Liu, X. F. et al. A clinical study on the resection of breast fibroadenoma using two types of incision. Scand. J. Surg. SJS Off. Organ Finn. Surg. Soc. Scand. Surg. Soc. 100, 147-152 (2011).
8. The Cancer Genome Atlas Network. Comprehensive molecular portraits of human breast tumours. Nature 490, 61-70 (2012).
9. Stephens, P. J. et al. The landscape of cancer genes and mutational processes in breast cancer. Nature 486, 400-404 (2012).
10. Millikan, R. et al. p53 mutations in benign breast tissue. J. Clin. Oncol. 13, 2293-2300 (1995).
11. Franco, N., Picard, S.-F., Mege, F., Arnould, L. & Lizard-Nacol, S. Absence of Genetic Abnormalities in Fibroadenomas of the Breast Determined at p53 Gene Mutations and Microsatellite Alterations. Cancer Res. 61, 7955-7958 (2001).
12. Vorkas, P. A. et al. PIK3CA Hotspot Mutation Scanning by a Novel and Highly Sensitive High-Resolution Small Amplicon Melting Analysis Method. J. Mol. Diagn. JMD 12, 697-704 (2010).
13. Vogelstein, B. et al. Cancer Genome Landscapes. Science 339, 1546-1558 (2013).
14. Mäkinen, N. et al. MED12, the mediator complex subunit 12 gene, is mutated at high frequency in uterine leiomyomas. Science 334, 252-255 (2011).
15. Forbes, S. A. et al. COSMIC: mining complete cancer genomes in the Catalogue of Somatic Mutations in Cancer. Nucleic Acids Res. 39, D945-D950 (2011).
16. Sherry, S. T. et al. dbSNP: the NCBI database of genetic variation. Nucleic Acids Res. 29, 308-311 (2001).
17. Consortium, T. 1000 G. P. An integrated map of genetic variation from 1,092 human genomes. Nature 491, 56-65 (2012).
18. Harper, P. S. Mary Lyon and the hypothesis of random X chromosome inactivation. Hum. Genet. 130, 169-174 (2011).
19. Subramanian, A. et al. Gene set enrichment analysis: A knowledge-based approach for interpreting genome-wide expression profiles. Proc. Natl. Acad. Sci. U.S.A. 102, 15545-15550 (2005).
20. Barbieri, C. E. et al. Exome sequencing identifies recurrent SPOP, FOXA1 and MED12 mutations in prostate cancer. Nat. Genet. 44, 685-689 (2012).
21. Assié, G. et al. Integrated genomic characterization of adrenocortical carcinoma. Nat. Genet. (2014). doi:10.1038/ng.2953
22. Network, T. C. G. A. R. Integrated genomic analyses of ovarian carcinoma. Nature 474, 609-615 (2011).
23. Je, E. M., Kim, M. R., Min, K. O., Yoo, N. J. & Lee, S. H. Mutational analysis of MED12 exon 2 in uterine leiomyoma and other common tumors. Int. J. Cancer 131, E1044-E1047 (2012).
24. Kämpjärvi, K. et al. Somatic MED12 mutations in uterine leiomyosarcoma and colorectal cancer. Br. J. Cancer 107, 1761-1765 (2012).
25. Zhu, B. T. & Conney, A. H. Functional role of estrogen metabolism in target cells: review and perspectives. Carcinogenesis 19, 1-27 (1998).
26. Kang, Y. K., Guermah, M., Yuan, C.-X. & Roeder, R. G. The TRAP/Mediator coactivator complex interacts directly with estrogen receptors α and β through the TRAP220 subunit and directly enhances estrogen receptor function in vitro. Proc. Natl. Acad. Sci. U.S.A. 99, 2642-2647 (2002).
27. Mäkinen, N., Vahteristo, P., Bützow, R., Sjöberg, J. & Aaltonen, L. A. Exomic landscape of MED12 mutation-negative and -positive uterine leiomyomas. Int. J. Cancer J. Int. Cancer 134, 1008-1012 (2014).
28. Chan-on, W. et al. Exome sequencing identifies distinct mutational patterns in liver fluke-related and non-infection-related bile duct cancers. Nat. Genet. 45, 1474-1478 (2013).
29. Li, H. & Durbin, R. Fast and accurate short read alignment with Burrows-Wheeler transform. Bioinforma. Oxf. Engl. 25, 1754-1760 (2009).
30. Li, H. et al. The Sequence Alignment/Map format and SAMtools. Bioinforma. Oxf. Engl. 25, 2078-2079 (2009).
31. Wilson, C. L. & Miller, C. J. Simpleaffy: a BioConductor package for Affymetrix Quality Control and data analysis. Bioinforma. Oxf. Engl. 21, 3683-3685 (2005).
32. Irizarry, R. A. et al. Exploration, normalization, and summaries of high density oligonucleotide array probe level data. Biostat. Oxf. Engl. 4, 249-264 (2003).
33. Dai, M. et al. Evolving gene/transcript definitions significantly alter the interpretation of GeneChip data. Nucleic Acids Res. 33, e175-e175 (2005).
34. Smyth, G. K. Linear models and empirical bayes methods for assessing differential expression in microarray experiments. Stat. Appl. Genet. Mol. Biol. 3, Article3 (2004).
35. Edgar, R., Domrachev, M. & Lash, A. E. Gene Expression Omnibus: NCBI gene expression and hybridization array data repository. Nucleic Acids Res. 30, 207-210 (2002).
36. Hänzelmann, S, Castelo, R. & Guinney, J. GSVA: gene set variation analysis for microarray and RNA-Seq data. BMC Bioinformatics 14, 7 (2013).
37. Jardim, D. L. F., Coney, A., & Subbiah, V. Comprehensive characterization of malignant phyllodes tumor by whole genomic and proteomic analysis: biological implications for targeted therapy opportunities. Orphanet Journal of Rare Diseases 8:112 (2013).

1. A method of assaying susceptibility and/or confirming diagnosis of breast fibroadenomas development in a human subject comprising:
performing a nucleic acid-based assay to analyze an isolated polynucleotide encoding at least exon 2 of MED12 gene from a sample acquired from the human subject; and
regarding the human subject with greater susceptibility and/or confirming diagnosis of breast fibroadenomas development by detecting a mutation in the isolated polynucleotide, wherein the mutation is a splice site mutation located at position −8 of exon 2 of the MED12 gene, a missense mutation located at codon 44 of cDNA of the MED12 gene or a missense mutation located at codon 36 of cDNA of the MED12 gene.

2. The method of claim 1, wherein the missense mutation is located at position 107 of codon 36 cDNA of the MED12 gene.

3. The method of claim 1, wherein the missense mutation is located at position 130 and/or 131 of codon 44 cDNA of the MED12 gene

4. The method of claim 1, wherein the missense mutation results in p.G44A, p.G44C, p.G44D, p.G44R, p.G44S, or p.G44V in a polypeptide translated from the MED12 gene.

5. The method of claim 1, wherein the performing a nucleic acid-based assay comprises sequencing the polynucleotide.

6. The method of claim 1, wherein the sample comprises stromal tissues.

7. The method of claim 1 further comprising the steps of detecting at least one mutation located at PIK3CA and/or TP53 gene of the subject upon detecting a mutation in the isolated polynucleotide encoding at least exon 2 of MED12 gene; and regarding developed fibroadenoma in the subject as benign state in the absence of detectable mutation located at PIK3CA and/or TP53 gene.
